(19) United States
(12) Patent Application Publication
(10) Pub. No.: US 2025/0258166 A1
(43) Pub. Date: Aug. 14, 2025

(54) MEANS AND METHODS FOR HIGH-THROUGHPUT GLYCOPROFILING OF PROTEINS

(71) Applicant: Glycanostics s.r.o., Bratislava – mestská cast Dúbravka (SK)

(72) Inventors: Jan TKAC, Bratislava – mestská cast Dúbravka (SK); Tomas Bertok, Bratislava – mestská cast Dúbravka (SK)

(21) Appl. No.: 18/856,381

(22) PCT Filed: Feb. 2, 2023
(86) PCT No.: PCT/EP2023/052586

§ 371 Date: Oct. 11, 2024

(30) Foreign Application Priority Data
Apr. 12, 2022 (EP) 22167920.2

(51) Int. Cl. G01N 33/543; G01N 33/574; G01N 33/68

(52) U.S. Cl. CPC G01N 33/54313; G01N 33/574; G01N 33/6896; G01N 2333/4709; G01N 2333/4724; G01N 2333/805; G01N 2333/8146; G01N 2333/96455; G01N 2440/38; G01N 2470/04

(57) ABSTRACT

The present invention discloses a method of determining the glycoprofile of a protein, comprising (a) contacting a sample comprising said protein with first beads having coupled thereto an antibody directed against said protein, to form an antibody-protein complex, (b) contacting said antibody-protein complex with one or more further beads, each further bead having coupled thereto (i) a label which amplifies a signal being generated and (ii) a lectin, to form an antibody-protein-lectin complex; and (c) determining the glycoprofile of said protein. Further disclosed are methods for diagnosing cancer, autoimmune diseases and inflammatory diseases as well as kits for performing the methods disclosed herein.

### CROSS-REFERENCE TO RELATED APPLICATIONS

The present application claims the benefit of priority of EP patent application Ser. No. 22/167,920.2 filed 12 Apr. 2022, the contents of which are hereby incorporated by reference in its entirety for all purposes.

### TECHNICAL FIELD OF THE INVENTION

The present invention discloses a method of determining the glycoprofile of a protein, comprising (a) contacting a sample comprising said protein with first beads having coupled thereto an antibody directed against said protein, to form an antibody-protein complex, (b) contacting said antibody-protein complex with one or more further beads, each further bead having coupled thereto (i) a label which amplifies a signal being generated and (ii) a lectin, to form an antibody-protein-lectin complex; and (c) determining the glycoprofile of said protein. Further disclosed are methods for diagnosing cancer, autoimmune diseases and inflammatory diseases as well as kits for performing the methods disclosed herein.

### BACKGROUND

Glycans are present on a variety of different proteins, where they have an impact on protein trafficking, stability and folding, ultimately altering its biochemical, and biophysical properties. Moreover, glycans can mediate proteolysis patterns or directly mediate ligand-receptor interactions, oncogenic signaling transduction, immune recognition, migration and both cell-cell and cell-matrix adhesion. As such, particular glycans may exert a selective advantage for tumor cells. The presence of particular glycans or the presence of particular glycans on particular proteins thus may be used as a biomarker, e.g., for the diagnosis of cancer.

Glycan structures can be analyzed by using binding molecules that specifically bind to a particular glycan structure. Besides antibodies specific for glycan structures also lectins can be employed. Lectins are carbohydrate-binding proteins that are highly specific for sugar groups that are part of other molecules. These binding molecules can be used in assays like enzyme-linked immunosorbent assay (ELISA), enzyme-linked lectin assay (ELLA), magnetic ELLA (MELLA) using optical, fluorescent luminescent or electrochemiluminescent reading or parallel/multiplexed Luminex-like assays to analyze the presence or absence of a particular glycan structure.

WO2019/185515 discloses a method for determining the glycoprofile of a protein of interest comprising the enrichment of said protein of interest with an antibody followed by contacting the complex thereby created with one or more lectins. Also Li et al. (2013), Clinical Chemistry, 59(1):315-324, disclose a method for determining the glycoprofile of a protein. Here, the protein of interest is enriched via antibodies coupled to beads and contacted with labelled lectins afterwards.

Carlstrom et al. (2018), Technical note. AlphaLISA Technology discloses a method for determining the glycoprofile of an antibody, wherein the antibody is bound by a lectin which itself is coupled to a bead (donor bead) and further being bound by a protein G coupled as modification to an AlphaLISA acceptor bead. Each bead is thus conjugated to said antibody and a detection of the glycosylation state of the antibody is thereby possible if the glycans bind to the lectin of the donor bead. When there is interaction, the donor bead is brought into proximity of the acceptor bead and excitation of the donor bead results in a luminescent signal from the acceptor bead (the Protein G AlphaLISA bead).

Additionally, Chen Li et al. (2011), Electrophoresis, 32(15): 2028-2035 discloses a method for profiling glycosylation patterns of proteins using a protein-specific capture antibody coupled to a bead. In such sandwich assay only one bead is applied—as detection reagent a fluorescent labeled lectin is used. The same applies mutatis mutandis to Jun Natsuki et al. (2005), Biotechnology and Bioengineering, 93(2): 225-230. In both prior art documents which comment on the antibody-lectin assay as quite a reliable tool for glycosylation profiling, there is no motivation or an incentive to even amend such antibody-lectin assays, since for example Chen Li et al. (2011) clearly emphasizes that future studies should be multiplexed with additional antibodies on the bead instead of changing the whole assay and using a second bead for the detection.

However, there still is an ongoing need for further improved methods for determining the glycoprofile of a protein. In particular, these methods should have a lower limit of detection, a higher sensitivity and selectivity of detection, particularly towards cancer-specific markers, preferably with shorter analysis time. The present invention aims to address this need.

### SUMMARY OF THE INVENTION

This need is solved by the subject-matter as defined in the claims and in the embodiments described herein.

Accordingly, the present invention relates to a method of determining the glycoprofile of a protein, comprising
(a) contacting a sample comprising said protein with first beads having coupled thereto an antibody directed against said protein, to form an antibody-protein complex,
(b) contacting said antibody-protein complex with one or more further beads, each further bead having coupled thereto (i) a label which amplifies a signal being generated and (ii) a lectin,
   to form an antibody-protein-lectin complex; and
(c) determining the glycoprofile of said protein.

The method of the invention may further comprise step (d) comparing the glycoprofile of said protein with a control glycoprofile of said protein to determine whether the glycoprofile of said protein may deviate from the glycoprofile of said control glycoprofile.

The method of the invention may further comprise step (a′) enriching said antibody-glycoprotein complex prior to step (b) contacting said antibody-glycoprotein complex with one or more further beads.

The method of the invention may further comprise step (b′) enriching said antibody-protein-lectin complex prior to step (c) determining the glycoprofile of said protein.

Preferably, said protein is a cancer biomarker protein, an autoimmune disease biomarker protein, an inflammatory disease biomarker protein or a neurodegenerative disease biomarker protein. Preferably, said protein is an autoimmune disease biomarker protein. Preferably, said protein is an inflammatory diseases biomarker. Preferably, said protein is a cancer biomarker protein, more preferably an ovarian cancer biomarker protein, breast cancer biomarker protein, colorectal cancer biomarker protein, pancreatic cancer biomarker protein, prostate cancer biomarker protein, thyroid cancer biomarker protein, liver cancer biomarker protein, lung cancer biomarker protein, stomach cancer biomarker protein, testicular cancer biomarker protein or bladder cancer biomarker protein. More preferably, said prostate cancer biomarker protein is β-haptoglobin, TIMP-1, PSA, fPSA or tPSA. Preferably, said protein is a neurodegenerative disease biomarker protein, more preferably α-synuclein, tau-protein or amyloid beta protein and its isoforms.

Preferably, said lectin is specific for core fucose, antennary fucose, Fucα1-6GlcNAc-N-Asn containing N-linked oligosaccharides, Fucα1-6/3GlcNAc, α-L-Fuc, Fucα1-2Galβ1-4(Fucα1-3)GlcNAc, Fucα1-2Gal, Fucα1-6GlcNAc, Manβ1-4GlcNAcβ1-4GlcNAc, branched N-linked hexa-saccharide, Manα1-3Man, α-D-Man, (GlcNAcβ1-4)₂₋₄, Galβ1-4GlcNAc, GlcNAcα1-4Galβ1-4GlcNAc, (GlcNAcβ1-4)₂₋₅, Neu5Ac (sialic acid), Galβ1-3GaINAc-serine/threonine, Galα1-3GalNAc, Galβ1-6Gal, Galβ1-4GlcNAc, Galβ1-3GalNAc, GalNAcα1-3GalNAc, GalNAcα1-3Gal, GalNAcα/β1-3/4Gal, α-GalNAc, GalNAcβ1-4Gal, GalNAcα1-3(Fucα1-2)Gal, GalNAcα1-2Gal, GalNAcα1-3GalNAc, GalNAcβ1-3/4Gal, GalNAc-Ser/Thr (Tn antigen), Galβ1-3GalNAc-Ser/Thr (T antigen), GalNAcβ1-4GlcNAc (LacdiNAc), α-2,3Neu5Ac (α2-3 linked sialic acid), α-2,6Neu5Ac (α2-6 linked sialic acid), α-2,8Neu5Ac (α2-8 linked sialic acid), sialic acid (α-2,3Neu5Ac, α-2,6Neu5Ac or α-2,8Neu5Ac), Neu5Acα4/9-O-Ac-Neu5Ac, Neu5Acα2-3Galβ1-4Glc/GlcNAc, Neu5Acα2-6Gal/GalNAc, N-linked bi-antennary, N-linked tri/tetra-antennary, branched β1-6GlcNAc, Galα1-3(Fucα1-2)Galβ1-3/4GlcNAc, Galβ1-3(Fucα1-4)GlcNAc, NeuAcα2-3Galβ1-3(Fucα1-4)GlcNAc, Fucα1-2Galβ1-3(Fucα1-4)GlcNAc, Galβ1-4(Fucα1-3)GlcNAc, NeuAcα2-3Galβ1-4(Fucα1-3)GlcNAc, Fucα1-2Galβ1-4(Fucα1-3)GlcNAc, high mannose, sialyl Lewis^{a} (sialyl Le^{a}) antigen, sialyl Lewis^{x} (sialyl Le^{x}) antigen, Lewis^{x} (Le^{x}) antigen, sialyl Tn antigen, sialyl T antigen, Lewis^{y} (Le^{y}) antigen, sulfated core1 glycan, Tn antigen, T antigen, core 2 glycan, Lewis^{a} (Le^{a}) antigen, (GlcNAcβ1-4)ₙ, β-D-GlcNAc, GalNAc, Gal-GlcNAc, GlcNAc, Galα1-3Gal, Galβ1-3GalNAc, α-Gal, α-GalNAc, (GlcNAc)ₙ, branched (LacNAc)ₙ.

The present invention may further comprise said method as defined elsewhere herein, wherein the protein is a cancer biomarker protein and wherein a deviation of said glycoprofile from a healthy glycoprofile of said cancer biomarker protein is indicative that said subject may be at a risk or may suffer from cancer.

The present invention may further comprise said method as defined elsewhere herein, wherein the protein is a autoimmune disease biomarker protein and wherein a deviation of said glycoprofile from a healthy glycoprofile of said autoimmune disease biomarker protein is indicative that said subject may be at a risk or may suffer from an autoimmune disease.

The present invention may further comprise said method as defined elsewhere herein, wherein the protein is a inflammatory disease biomarker protein and wherein a deviation of said glycoprofile from a healthy glycoprofile of said inflammatory disease biomarker protein is indicative that said subject may be at a risk or may suffer from an inflammatory disease.

The present invention may further comprise said method as defined elsewhere herein, wherein the protein is a neurodegenerative disease biomarker protein and wherein a deviation of said glycoprofile from a healthy glycoprofile of said neurodegenerative disease biomarker protein is indicative that said subject may be at a risk or may suffer from a neurodegenerative disease.

The present invention further relates to a kit for performing the method for diagnosing whether a subject may be at a risk or may suffer from cancer of the invention comprising an antibody specific for a cancer biomarker protein as defined herein and one or more lectins as defined herein.

The present invention further relates to a kit for performing the method for diagnosing whether a subject may be at a risk or may suffer from an autoimmune disease of the invention, comprising an antibody specific for an autoimmune disease biomarker protein which is IgG and one or more lectins as defined herein.

The present invention further relates to a kit for performing the method for diagnosing whether a subject may be at a risk or may suffer from an inflammatory disease of the invention, comprising an antibody specific for an inflammatory biomarker protein which is IgG, IgA or CRP and one or more lectins as defined herein.

The present invention further relates to a kit for performing the method for diagnosing whether a subject may be at a risk or may suffer from a neurodegenerative disease, comprising an antibody specific for a neurodegenerative biomarker protein, which is α-synuclein, tau-protein or amyloid beta protein and its isoforms, and one or more lectins as defined herein.

Preferably, said first beads and said further beads are simultaneously brought into contact with said sample.

Preferably, said further beads are brought into contact with said sample immediately after said first beads were brought into contact with said sample.

Preferably, said first beads are brought into contact with said sample immediately after said second beads were brought into contact with said sample.

Preferably, said first bead and said further beads are in solution during performing the method of any one of the preceding claims.

Preferably, said first bead and/or said further beads is/are made of glass, plastic, metal, agarose, latex, metallic nano- or microparticle, metal oxide nano- or microparticle or magnetic material.

Preferably, the label of said further beads is an enzyme, a radioisotope, a fluorescent protein, a fluorescent dye, a bioluminescent label or a tag (e.g., biotin).

The label of said one or more further beads may be detected based on optical, fluorescent, luminescent, electrochemiluminescent and/or multi-analyte profiling (xMAP) readouts or means.

Preferably, for each of the one or more further beads for each carbohydrate detected by a lectin a different label is used in combination.

### BRIEF DESCRIPTION OF THE DRAWINGS

The invention will be better understood with reference to the detailed description when considered in conjunction with the non-limiting examples and the accompanying drawings, in which:

FIG. 1 depicts an exemplary scheme of an exemplary embodiment of the invention. Depicted are the first bead (B1), a further bead (B2), and said protein (analyte).

FIG. 2 depicts the depiction of the assay configuration for high-throughput fPSA glycoprofiling (without the need for any washing steps). Anti-fPSA modified magnetic nanoparticles (1) are being mixed with a sample (2) containing analyte i.e. fPSA in the first chamber, subsequently being held at the bottom of the second chamber by a magnetic field. Through this second chamber, lectin/peroxidase-modified nanoparticles are being introduced at first (3), subsequently being washed away by a substrate solution (4). After a short period of time, the solution is being pumped into a spectrophotometer chamber (5) and signal—change in colour, is detected. Inset: ROC curve showing the difference between tPSA (black line) and PGI+ index (light grey line) in this arrangement, with AUC values of 0.686 (Cl95%=[0.581;0.781], specificity=0.654, sensitivity=0.690, accuracy=0.673) for tPSA and 0.803 (Cl95%=[0.707; 0.890], specificity=0.712, sensitivity=0.828, accuracy=0.773) for PGI+, respectively. The amount of samples used in the study was 110 in total (58 BPH and 52 PCa patients).

### DETAILED DESCRIPTION OF THE INVENTION

The present invention is described in detail in the following and will also be further illustrated by the appended examples and figures.

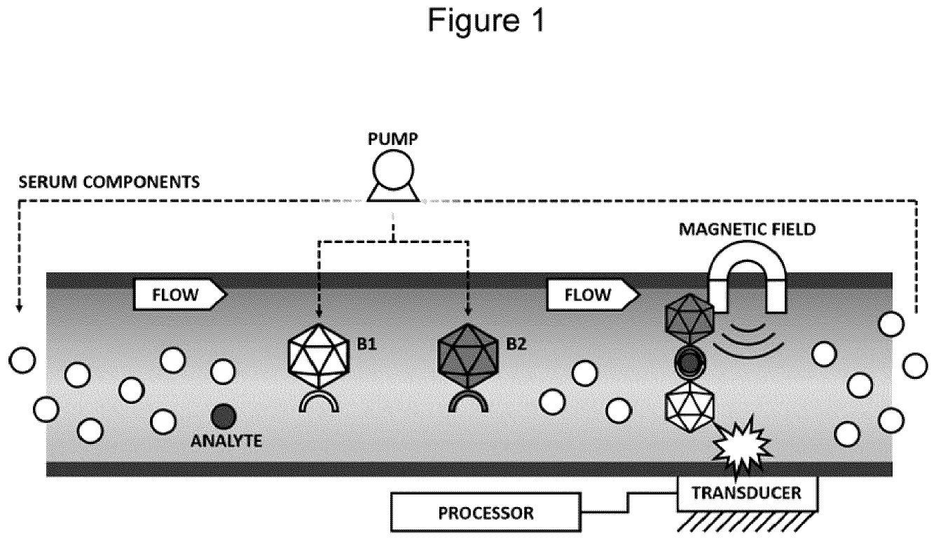

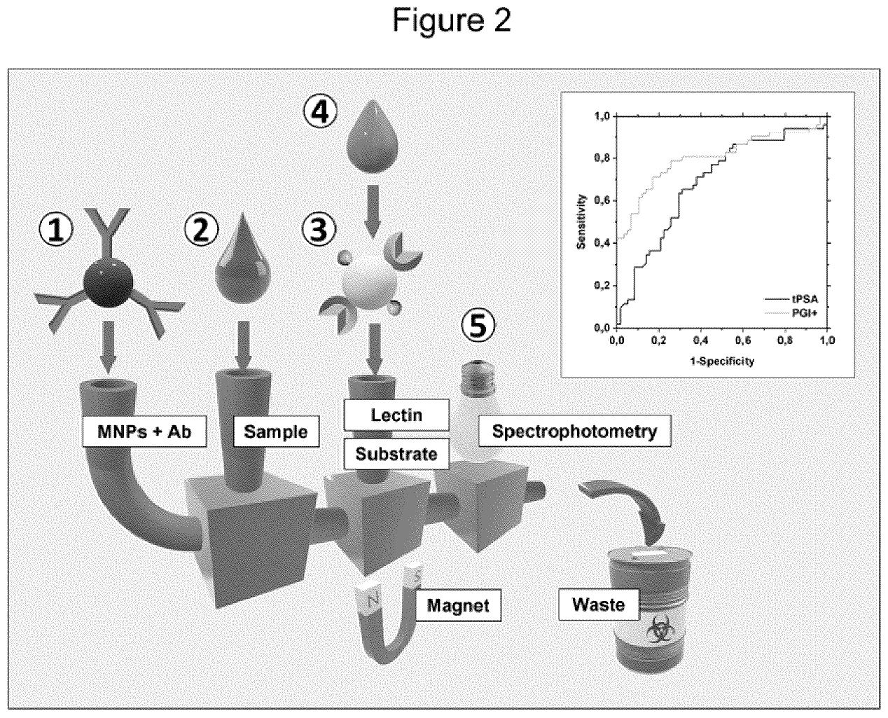

As outlined above, the present invention describes a method of determining the glycoprofile of a protein (of interest). This method can be described in an exemplary embodiment as follows (see also FIGS. 1 and 2, or Example 1 or 2). First, a sample comprising the protein (of interest, “analyte” in FIG. 1; fPSA in FIG. 2) is contacted with first beads (“B1” in FIG. 1; “MNPs +Ab” in FIG. 2) having coupled thereto an antibody directed against said protein. Thus, the protein is captured on the first beads. The protein may but does not have to be enriched, e.g., by applying a magnet in case the first beads are magnetic and washing away unbound proteins. Afterwards, the first bead-antibody-protein complex is contacted with one or more further beads (“B2” in FIG. 1; nanoparticle comprising HRP and lectin in FIG. 2). These further beads comprise a lectin specifically binding to the glycans of the protein and a label. Hence, the further beads only bind to the first bead-antibody-protein complex in case the protein carries a glycan specifically bound by said lectin, or in other words, has a glycoprofile detected by said lectin. Thus, even if only one protein having the glycan structure of interest/bound by the lectin is bound to the first bead, it can be bound by the lectin on the one or more further beads, which themselves comprise a label, ideally a plurality of labels. All these labels on the further beads are active and provide a signal—or a processed signal even if only one lectin binds. This can be described as an amplification effect. Thus, the combination of lectins and labels on a single (further/second) bead allows for a very low level of detection as shown in the Examples. Further advantages are a higher sensitivity and selectivity of detection, in particular regarding cancer-specific biomarkers. Additionally, the time needed for analysis is reduced. In Carlstrom et al. (2018)—which sandwich assay also clearly distinguishes to the method of the invention that instead of a protein G an antibody being directed to a glycoprotein is used—there is no such amplification effect which is according to the invention due to the label coupled to the further/second bead also comprising lectin as described above. In Carlstrom et al. (2018) a detectable signal is just generated by the acceptor bead which is coupled to the Protein G (such bead does also not comprise any lectin).

Although both WO 2019/185515 and Li et al. (2013), Clinical Chemistry, 59(1):315-324 disclose methods for determining the glycoprofile both fail to describe one or more further beads, each further bead having coupled thereto (i) a label and (ii) a lectin.

Accordingly, the present invention relates to a method of determining the glycoprofile of a protein, comprising
(a) contacting a sample comprising said protein with first beads having coupled thereto an antibody directed against said protein,
   to form an antibody-protein complex,
(b) contacting said antibody-protein complex with one or more further beads, each further bead having coupled thereto (i) a label and (ii) a lectin,
   to form an antibody-protein-lectin complex; and
(c) determining the glycoprofile of said protein.

The term “glycoprofile of a protein” means a carbohydrate structure of the protein (of interest), e.g., composition and/or structure of covalently linked carbohydrates, e.g., quantity, presence, or absence of covalently linked carbohydrates. The term “glycoprofiling” or “determining of a glycoprofile” means determining a carbohydrate structure (e.g., composition and/or structure of covalently linked carbohydrates, e.g., quantity, presence, or absence of covalently linked carbohydrates) on said protein (of interest).

The method of the present invention may be used to determine whether a protein has a particular glycoprofile or, in other words, carry a specific glycan. This can also be used to differentiate whether the protein carries a glycan that is indicative for a disease or not. Thus, a protein with a control glycoprofile, i.e. a protein having a known glycoprofile, can be used as a standard or positive control and be compared to the signal obtained by said protein, e.g., present in a sample (obtained from a subject). In case there is a deviation, this might be indicative for another glycoprofile. Accordingly, the method of determining the glycoprofile of a protein of the invention may further comprise step (d) comparing the glycoprofile of said protein with a control glycoprofile of said protein to determine whether the glycoprofile of said protein may deviate from the glycoprofile of said control glycoprofile.

The present invention is however not limited to the detection of only one particular glycan structure but can be used to detect two, three, four, five or more than five different glycan structures. In this case, “glycoprofiling” includes the determination of more than one such as two, three, four, five or more than five different carbohydrate structure (e.g., composition and/or structure of covalently linked carbohydrates, e.g., quantity, presence, or absence of covalently linked carbohydrates) on a protein of interest. Advantageously, different labels are used for each different glycan structure to be analyzed. Thereby, particular pairs of one particular lectin and one particular label coupled to one particular further bead are provided. Also different modes of detection may be combined, e.g., based on fluorescent, luminescent or chemiluminescent labels providing corresponding signals. Accordingly, for each of the one or more further beads for each carbohydrate detected by a lectin a different label preferably is used in combination.

In the methods of the invention, the first beads and/or the one or more further beads can be enriched. This can be used to even further reduce background signals. “Enriching” as used herein describes the process of increasing the amount of the bead/complex/substance in a mixture. Accordingly, the method of determining the glycoprofile of a protein of the invention may further comprise step (a′) enriching said antibody-glycoprotein complex prior to step (b) contacting said antibody-glycoprotein complex with one or more further beads. Furthermore, the method of determining the glycoprofile of a protein of the invention may further comprise step (b′) enriching said antibody-protein-lectin complex prior to step (c) determining the glycoprofile of said protein. To be clear and only to state the obvious, said steps (a′) and/or (b′) may also be added in the methods for diagnosing whether a subject may be at a risk or may suffer from cancer, for diagnosing whether a subject may be at a risk or may suffer from an autoimmune disease or for diagnosing whether a subject may be at a risk or may suffer from an inflammatory disease of the invention or for diagnosing whether a subject may be at risk or may suffer from a neurodegenerative disease.

The robustness of the present invention allows that the first beads and the further beads can be simultaneously or immediately consecutively brought into contact with the sample. Accordingly, said first beads and said further beads may be simultaneously brought into contact with said sample. Alternatively, said further beads may be brought into contact with said sample immediately after said first beads were brought into contact with said sample. Alternatively, said first beads can be brought into contact with said sample immediately after said second beads were brought into contact with said sample. This also means that the order of steps (a) and (b) is not necessarily set by their designation of step (a) and (b). However, step (a) followed by step (b) followed by step (c) is preferred.

Additionally, said first bead and said further beads preferably are in solution during performing the methods described herein. “In solution” in this context means that neither said first beads nor said further beads are particularly not hold by a magnetic force nor coupled to a solid material such as a microplate, a column or a reaction tube. In one embodiment, said first bead is not hold by a magnetic force nor coupled to a solid material such as a microplate, a column or a reaction tube. In one embodiment, said one or more further bead is not hold by a magnetic force nor coupled to a solid material such as a microplate, a column or a reaction tube.

The protein (to be glycoprofiled by the methods of the invention) is not particularly limited. However, the protein preferably is a glycoprotein. Since the presence or absence of a particular glycan structure on the protein may be important for diagnosis or prognosis of a disease, the protein of interest preferably is a protein, whose glycoprofile is relevant for a disease. The term “glycoprotein” (or “glycosylated protein”) as used herein means a protein containing one or more N—, O—, S— or C— covalently linked carbohydrates of various types, e.g., ranging from monosaccharides to branched oligosaccharides or polysaccharides (including their modifications such as sulfo- or phospho-group attachment). N-linked glycans are carbohydrates bound to —NH₂ group of asparagine. O-linked glycans are carbohydrates bound to —OH group of serine, threonine, or hydroxylated amino acids. S-linked glycans are carbohydrates bound to —SH group of cysteine. C-linked glycans are carbohydrates bound to tryptophan via C—C bond.

The term “carbohydrates” means compounds (e.g., such as aldoses and ketoses) having the stoichiometric formula Cₙ(H₂O)ₙ. The generic term “carbohydrate” includes monosaccharides, oligosaccharides and polysaccharides as well as substances derived from monosaccharides by reduction of the carbonyl group (alditols), by oxidation of one or more terminal groups to carboxylic acids, or by replacement of one or more hydroxy group(s) by a hydrogen atom, an amino group, thiol group or similar groups. It also includes derivatives of these compounds.

As already described herein, the presence of a particular glycoprofile or glycan on the protein of interest may be relevant for the diagnosis of a particular disease such as a cancer, an autoimmune disease, an inflammatory disease or a neurodegenerative disease. Some combinations of proteins (of interest, i.e. biomarker proteins) and glycan structures (A) are known to be indicative for diseases. Specific combinations of proteins (of interest) and glycans indicative for diseases are exemplified in Fehler! Verweisquelle konnte nicht gefunden werden. as well as antibodies and lectins binding to the particular glycan structures. Thus, the method and uses of the present invention can be used in diagnosing of diseases such as cancer, autoimmune disease, inflammatory disease, or neurodegenerative disease.

Accordingly, the presence of said particular glycoprofile or glycan structure may be indicative of a disease such as cancer, autoimmune disease, inflammatory disease, or neurodegenerative disease.

In this context, the protein preferably is a cancer biomarker protein, an autoimmune disease biomarker protein, an inflammatory disease biomarker protein, or a neurodegenerative disease biomarker protein. More preferably, the protein preferably is a cancer biomarker protein. More preferably, the protein preferably is an autoimmune disease biomarker protein. More preferably, the protein preferably is an inflammatory disease biomarker protein. More preferably, the protein preferably is a neurodegenerative disease biomarker protein.

As used herein, an “autoimmune disease” refers a group of diseases characterized by disease associated with the production of antibodies directed against one's own tissues. Non-limiting examples of an autoimmune disease include, but are not limited to, Hashimoto's disease, primary biliary cirrhosis, systemic lupus erythematosus, rheumatic fever, rheumatoid arthritis, autoimmune hemolytic anemia, idiopathic thrombocytopenia purpura, and post viral encephalomyelitis, Addison's disease, autoimmune enteropathy, primary biliary cirrhosis, Goodpasture's syndrome, Hashimoto's thyroiditis, myasthenia gravis, myxoedema, pemphigoid, rheumatoid arthritis, Sjogren's syndrome, symphathetic ophthalmitis, both forms of lupus erythematosus, thyrotoxicosis, ulcerative colitis, multiple sclerosis, celiac disease, diabetes mellitus type 1, Graves' disease, inflammatory bowel disease and psoriasis.

As used herein, an “inflammatory disease” refers a group of diseases characterized by impairment and/or abnormal functioning of inflammatory mechanisms of the body. Non-limiting examples of an inflammatory disease include, but are not limited to, necrotizing enterocolitis, gastroenteritis, pelvic inflammatory disease (PID), empyema, pleurisy, pyelitis, pharyngitis, angina, arthritis, acne, urinary tract infections, Acne vulgaris, Asthma, Celiac disease, Chronic prostatitis, Colitis, Diverticulitis, Glomerulonephritis, Hidradenitis suppurativa, Hypersensitivities, Inflammatory bowel diseases, Interstitial cystitis, Mast Cell Activation Syndrome, Mastocytosis, Otitis, Pelvic inflammatory disease, Reperfusion injury, Rheumatic fever, Rheumatoid arthritis, Rhinitis, Sarcoidosis, Transplant rejection, Vasculitis.

As used herein, a “neurodegenerative disease” refers a group of diseases characterized by impairment and/or abnormal functioning of brain. Non-limiting examples of an inflammatory disease include, Parkinson disease, Alzheimer disease and other forms of tautopathy diseases Primary age-related tauopathy, Chronic traumatic encephalopathy, Progressive supranuclear palsy, Corticobasal degeneration, Frontotemporal dementia and parkinsonism linked to chromosome 17, Vacuolar tauopathy, Lytico-bodig disease, Ganglioglioma and gangliocytoma, Meningioangiomatosis, Postencephalitic parkinsonism, Subacute sclerosing panencephalitis, lead encephalopathy, tuberous sclerosis, Pantothenate kinase-associated neurodegeneration, and lipofuscinosis, etc.

As used herein, “cancer” refers a group of diseases characterized by the uncontrolled growth of abnormal cells in the body. Unregulated cell division may result in the formation of malignant tumours or cells that invade neighbouring tissues and may metastasize to distant parts of the body through the lymphatic system or bloodstream. Non-limiting examples of cancers include squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumour, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the oesophagus, cancer of the small intestine, cancer of the endocrine system, cancer of the parathyroid gland, cancer of the adrenal gland, sarcoma of soft tissue, cancer of the urethra, cancer of the penis, solid tumours of childhood, cancer of the ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumour), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukaemia, lymphomas, and myelomas, e.g., acute, chronic, lymphocytic and/or myelogenous leukaemia, such as acute leukaemia (ALL), acute myelogenous leukaemia (AML), chronic lymphocytic leukaemia (CLL), and chronic myelogenous leukaemia (CML), undifferentiated AML (MO), myeloblastic leukaemia (M1), myeloblastic leukaemia (M2; with cell maturation), promyelocytic leukaemia (M3 or M3 variant [M3V]), myelomonocytic leukemia (M4 or M4 variant with eosinophilia [M4E]), monocytic leukaemia (M5), erythroleukaemia (M6), megakaryoblastic leukaemia (M7), isolated granulocytic sarcoma, and chloroma; lymphomas, such as Hodgkin' s lymphoma (HL), non-Hodgkin' s lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukaemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation, lymphoproliferative disorder, true histiocytic lymphoma, primary central nervous system lymphoma, primary effusion lymphoma, lymphoblastic lymphoma (LBL), hematopoietic tumours of lymphoid lineage, acute lymphoblastic leukaemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, non-secretory myeloma, smouldering myeloma (also called indolent myeloma), solitary, plasmocytoma, and multiple myelomas, chronic lymphocytic leukaemia (CLL), hairy cell lymphoma; hematopoietic tumours of myeloid lineage, tumours of mesenchymal origin, including fibrosarcoma and rhabdomyosarcoma; seminoma, teratocarcinoma, tumours of the central and peripheral nervous, including astrocytoma, schwannomas; tumours of mesenchymal origin, including fibrosarcoma, rhabdomyosarcoma, and osteosarcoma; and other tumours, including melanoma, xeroderma pigmentosum, keratoacanthoma, seminoma, thyroid follicular cancer and teratocarcinoma, hematopoietic tumours of lymphoid lineage, for example T-cell and B-cell tumours, including but not limited to T-cell disorders such as T-prolymphocytic leukaemia (T-PLL), including of the small cell and cerebriform cell type; large granular lymphocyte leukaemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. Preferred cancers are also shown in Fehler! Verweisquelle konnte nicht gefunden werden.

The cancer may also be an ovarian cancer, breast cancer, colorectal cancer, pancreatic cancer, prostate cancer, thyroid cancer, liver cancer, lung cancer, stomach cancer, testicular cancer or bladder cancer. Accordingly, the biomarker protein (of interest) may be an ovarian cancer biomarker protein, breast cancer biomarker protein, colorectal cancer biomarker protein, pancreatic cancer biomarker protein, prostate cancer biomarker protein, thyroid cancer biomarker protein, liver cancer biomarker protein, lung cancer biomarker protein, stomach cancer biomarker protein, testicular cancer biomarker protein or bladder cancer biomarker protein.

Exemplary cancers, cancer biomarkers with aberrant glycosylation, lectins, antibodies and corresponding glycan modifications within the meaning of the present invention are also shown in Fehler! Verweisquelle konnte nicht gefunden werden. below. Lectin abbreviations used in Fehler! Verweisquelle konnte nicht gefunden werden.: AAA—Anguilla anguilla agglutinin (UniProtKB Accession Number: Q7SIC1), AAL—Aleuria aurantia lectin, ABA—Agaricus bisporus agglutinin, ACA—Amaranthus caudatus agglutinin, AHA—Arachis hypogaea agglutinin=peanut agglutinin (PNA), AlA—Artocarpus integrifolia agglutinin=Jacalin, AlloA—Allomyrina dichotoma agglutinin, AOL—Aspergillus oryzae lectin, BanLec—Musa paradisiaca lectin, BS-I—Bandeiraea simplicifolia lectin=Griffonia (Bandeiraea) simplicifolia lectin I, Con A—Concanavalin A, DBA—Dolichos biflorus agglutinin, DSA—Datura stramonium agglutinin (Jacalin), ECL—Erythrina cristagalli lectin, GNA—Galanthus nivalis agglutinin, GSA I (GSL I)—Griffonia (Bandeiraea) simplicifolia lectin I, GSL II—Griffonia (Bandeiraea) simplicifolia lectin II, HHL—Hippeastrum hybrid (Amaryllis) lectin, HPA—Helix pomatia agglutinin, LBA—Phaseolus lunatus (lima bean, LBA), LEL—Lycopersicon esculentum (tomato) lectin, LCA—Lens culinaris agglutinin, LTA—Lotus tetragonolobus lectin, MAA I—Maackia amurensis agglutinin I, MAA II—Maackia amurensis agglutinin II, MGBL 1—macrophage galactose binding lectin 1, MGBL 2 (macrophage galactose binding lectin 2, NPA—Narcissus pseudonarcissus (Daffodil) lectin, PHA E—Phaseolus vulgaris agglutinin E, PHA L—Phaseolus vulgaris agglutinin L, PhoSL—Pholiota squarrosa lectin, PNA—Peanut agglutinin, PSL—Pisum sativum lectin, PTA I—Psophocarpus tetragonolobus lectin I, PTA II—Psophocarpus tetragonolobus II, PWM—Phytolacca americana, RCA I—Ricinus communis agglutinin I, RCA II—Ricinus communis agglutinin II, SBA—Soybean agglutinin (Glycine max agglutinin), SCA—Sambucus canadensis agglutinin=Sambucus nigra agglutinin (SNA), SJA—Sophora japonica agglutinin II, SNA—Sambucus nigra agglutinin, SSA—Sambucus sieboldiana agglutinin, SSL—Salvia sclarea lectin, STL—Solanum tuberosum lectin, TJA-I—Trichosanthes japonica agglutinin I, TJA-II—Trichosanthes japonica agglutinin (Yamashita et al.), TVA—Triticum vulgaris agglutinin=WGA—wheat germ agglutinin, UEA—Ulex europaeus agglutinin, VVA—Vicia villosa lectin, WFA—Wisteria floribunda lectin, WGA—wheat germ agglutinin=TVA—Triticum vulgaris agglutinin. The symbol “†”, an upward pointing arrow means increase in concentration of a corresponding glycan/s or a complex/s (e.g., dimer, trimer etc). The symbol “↓”, a downward pointing arrow means increase in concentration of a corresponding glycan/s or a complex/s (e.g., dimer, trimer etc).

TABLE 1
Cancers, corresponding cancer biomarkers with aberrant glycosylation, lectins
and antibodies. The combinations of this table are merely examples for different cancer types.
The present invention is not limited to these exemplary combinations.

| | | | | | (Other) |
|---|---|---|---|---|---|
| | | Glycan | Lectins/antibodies | | applicable |
| Cancer | Biomarker | modification | applied | Refs. | lectins/Abs |
| Prostate | Prostate specific | ↑ α2-3Neu5Ac | MAA | [1-5] | anti-α2-3- |
| | antigen (PSA) | | | | linked sialic |
| | | | | | acid |
| | | | | | antibody |
| | | | | | (i.e. i.e. |
| | | | | | HYB4), |
| | | | | | Siglec 1, |
| | | | | | Siglec 4 or |
| | | | | | Siglec 8 |
| | fPSA | ↑ α2-3Neu5Ac | SNA* | [6, 7] | anti-α2,3- |
| | | | (determination of | | linked sialic |
| | | | non-eluted PSA | | acid |
| | | | from SNA affinity | | antibody |
| | | | column) | | (i.e. |
| | | | | | HYB4i.e. |
| | | | | | i.e. HYB4), |
| | | | | | MAA, |
| | | | | | Siglec 1, |
| | | | | | Siglec 4 or |
| | | | | | Siglec 8 |
| | fPSA | ↑ α2-3 Neu5Ac | anti-a2-3-linked | [8] | MAA, |
| | | | sialic acid antibody | | Siglec 1, |
| | | | (i.e. HYB4) | | Siglec 4 or |
| | | | | | Siglec 8 |
| | PSA [1], | ↓ bi-antennary | Con A | [1, 9] | |
| | tPSA/fPSA [9] | glycans | | | |
| | PSA [1], | ↓ high | Con A | [1, 9] | GNA, NPA |
| | tPSA/fPSA [9] | mannose | | | |
| | | glycans | | | |
| | PSA | ↓ α2-6Neu5Ac | SNA | [1] | TJA-I, SCA |
| | PSA | ↓ α2-6Neu5Ac | TJA-I | [2] | SNA, SCA |
| | PSA | ↑ tri-, tetra- | DSA (Jacalin) | [2] | PHA-L, |
| | | antennary | | | PHA-E |
| | | glycans | | | |
| | PSA | ↑ α1-2fucose, | TJA-II | [2] | AAL, UEA-I, |
| | | GalNAc | | | LCA, PSL, |
| | | | | | AAA, LTA, |
| | | | | | HPA, LBA, |
| | | | | | WFA, VVA |
| | PSA [2], | ↑ α1-2fucose | UEA-I | [2] | TJA II, AAL, |
| | fPSA/tPSA [10] | | | [10] | LCA, PSL, |
| | | | | | AAA, LTA |
| | PSA [2], tPSA [11, | ↑ LacdiNAc, | WFA | [2, 11, | DBA, SBA, |
| | 12] | GalNAc | | 12] | HPA, LBA, |
| | | | | | VVA |
| | tPSA | ↑ α1-3/6fucose | AAL | [13] | TJA II, |
| | | | | | UEA-I, |
| | | | | | LCA, PSL, |
| | | | | | AAA, LTA, |
| | | | | | AOL, |
| | | | | | PhoSL |
| | PSA in urine | ↓ α1-3/6 | AAL | [14] | TJA II, |
| | | fucose | | | UEA-I, |
| | | | | | LCA, PSL, |
| | | | | | AAA, LTA. |
| | | | | | AOL, |
| | | | | | PhoSL |
| | PSA in urine | ↓ core fucose | PhoSL | [14] | AOL |
| | | (α1-6fucose) | | | |
| | fPSA | ↓ core fucose | PhoSL | [6] | AOL |
| | | (α1-6fucose) | | | |
| | Tissue inhibitor of | ↑ α1-3/6fucose | AAL | [13] | AOL, |
| | metallopeptidase 1 | | | | PhoSL, TJA |
| | (TIMP1) | | | | II, UEA-I, |
| | | | | | LCA, PSL, |
| | | | | | AAA, LTA |
| | β-haptoglobin | ↑ core fucose | No lectin used, but | [15] | PhoSL, |
| | | (α1-6fucose) | MS | | AOL |
| | β-haptoglobin | ↑ | AAL | [16, | AOL, |
| | | core/antennary | | 17] | PhoSL, TJA |
| | | fucose | | | II, UEA-I, |
| | | | | | LCA, PSL, |
| | | | | | AAA, LTA |
| | β-haptoglobin | ↑ a2-6Neu5Ac | SNA | [16, | TJA-I, SCA |
| | | | | 17] | |
| | β-haptoglobin | ↑ tri-,tetra- | PHA-L | [16, | PHA-E, |
| | | antennary | | 17] | DSA |
| | | glycans | | | (Jacalin) |
| | β-haptoglobin | ↑ sialyl Lewis^{a} | Antibody against | [16] | SNA, TJA-I, |
| | | glycan | sialyl Lewis^{a} glycan | | MAA, anti- |
| | | | | | α2-3-linked |
| | | | | | sialic acid I |
| | | | | | antibody |
| | | | | | (i.e. |
| | | | | | HYB4i.e. |
| | | | | | i.e. HYB4), |
| | | | | | Siglec 1, |
| | | | | | Siglec 4 or |
| | | | | | Siglec 8 |
| | β-haptoglobin | ↑ sialyl Lewis^{x} | Antibody against | [17] | SNA, TJA-I, |
| | | glycan | sialyl Lewis^{x} glycan | | MAA, anti- |
| | | | | | α2-3-linked |
| | | | | | sialic acid |
| | | | | | antibody |
| | | | | | (i.e. |
| | | | | | HYB4i.e. |
| | | | | | i.e. HYB4), |
| | | | | | Siglec 1, |
| | | | | | Siglec 4 or |
| | | | | | Siglec 8 |
| | β-haptoglobin | ↑ antennary | No lectin used, but | [18] | TJA II, AAL, |
| | | fucose | MS | | UEA-I, |
| | | | | | LCA, PSL, |
| | | | | | AAA, LTA |
| | β-haptoglobin | ↑ tri-,tetra- | No lectin used, but | [18] | PHA-L, |
| | | antennary | MS | | PHA-E, |
| | | glycans | | | DSA |
| | β-haptoglobin | ↑ sialyl Lewisª | No lectin used, but | [18] | Antibodies |
| | | and sialyl | MS | | against |
| | | Lewis^{x} glycans | | | sialyl |
| | | | | | Lewisª and |
| | | | | | Lewis^{x} |
| | | | | | glycans, |
| | | | | | SNA, TJA-I, |
| | | | | | MAA, anti- |
| | | | | | α2-3-linked |
| | | | | | sialic acid |
| | | | | | antibody |
| | | | | | (i.e. |
| | | | | | HYB4i.e. |
| | | | | | i.e. HYB4), |
| | | | | | Siglec 1, |
| | | | | | Siglec 4 or |
| | | | | | Siglec 8 |
| | β-haptoglobin | ↑ antennary | AAL | [19] | TJA II, |
| | | fucose | | | UEA-I, |
| | | | | | LCA, PSL, |
| | | | | | AAA, LTA |
| Ovarian | α₁-acid | ↑ tri-,tetra- | Capillary | [20] | PHA-L, |
| | glycoprotein | antennary | electrophoresis | | PHA-E, |
| | | glycans | (CE) | | DSA |
| | α₁-acid | ↑ core fucose | CE | [20] | PhoSL, |
| | glycoprotein | | | | AOL |
| | α₁-acid | ↑ α2-6Neu5Ac | 2D PAGE and LC | [21] | TJA-I, SNA |
| | glycoprotein | | | | |
| | α₁-acid | ↑ sialyl Le^{x} | 2D PAGE and LC | [21] | Antibody |
| | glycoprotein | | | | against |
| | | | | | sLe^{x}, SNA, |
| | | | | | TJA-I, MAA, |
| | | | | | anti-α2-3- |
| | | | | | linked sialic |
| | | | | | acid |
| | | | | | antibody |
| | | | | | (i.e. |
| | | | | | HYB4i.e. |
| | | | | | i.e. HYB4), |
| | | | | | Siglec 1, |
| | | | | | Siglec 4 or |
| | | | | | Siglec 8 |
| | α₁-acid | ↓ α2-3Neu5Ac | 2D PAGE and LC | [21] | MAA, anti- |
| | glycoprotein | | | | α2-3-linked |
| | | | | | sialic acid |
| | | | | | antibody |
| | | | | | (i.e. |
| | | | | | HYB4i.e. |
| | | | | | i.e. HYB4), |
| | | | | | Siglec 1, |
| | | | | | Siglec 4 or |
| | | | | | Siglec 8 |
| | C1 esterase | ↑ Le^{x} | CE | [20] | Antibody |
| | inhibitor | | | | against Le^{x}, |
| | | | | | LTA |
| | C1 esterase | ↑ tri-antennary | CE | [20] | DBA, PHA- |
| | inhibitor | glycans | | | E, PHA-L |
| | 2-HS glycoprotein | ↑ tri-,tetra- | CE | [20] | DBA, PHA- |
| | | antennary | | | E, PHA-L |
| | | glycans | | | |
| | β-haptoglobin | ↑ tri-,tetra- | CE | [20] | DBA, PHA- |
| | | antennary | | | E, PHA-L |
| | | glycans | | | |
| | β-haptoglobin | ↑ Le^{x} | CE | [20] | Antibody |
| | | | | | against Le^{x}, |
| | | | | | LTA |
| | β-haptoglobin | ↑ sialyl Le^{x} | 2D PAGE and LC | [21] | Antibody |
| | | | | | against |
| | | | | | sLe^{x}, SNA, |
| | | | | | TJA-I, MAA, |
| | | | | | anti-α2-3- |
| | | | | | linked sialic |
| | | | | | acid |
| | | | | | antibody |
| | | | | | (i.e. |
| | | | | | HYB4i.e. |
| | | | | | i.e. HYB4), |
| | | | | | Siglec 1, |
| | | | | | Siglec 4 or |
| | | | | | Siglec 8 |
| | β-haptoglobin | ↑ α2-6Neu5Ac | 2D PAGE and LC | [21] | SNA, TJA-I |
| | β-haptoglobin | ↓ α2-3Neu5Ac | 2D PAGE and LC | [21] | MAA, anti- |
| | | | | | α2-3-linked |
| | | | | | sialic acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | β-haptoglobin | ↑ tri-, tetra- | LTA affinity | [22] | DBA, PHA- |
| | | antennary | separation AND | | E, PHA-L |
| | | glycans | PAGE | | |
| | β-haptoglobin | ↑ α2-3Neu5Ac | LTA affinity | [22] | MAA, anti- |
| | | | separation AND | | α2-3-linked |
| | | | PAGE | | sialic acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | β-haptoglobin | ↓ α2-6Neu5Ac | LTA affinity | [22] | SNA, TJA-I |
| | | | separation AND | | |
| | | | PAGE | | |
| | β-haptoglobin | ↑ antennary | LTA affinity | [22] | TJA II, AAL, |
| | | fucose | separation AND | | UEA-I, |
| | | | PAGE | | LCA, PSL, |
| | | | | | AAA, AAL |
| | β-haptoglobin | ↓ bi-antennary | Con A | [22] | NPA, GNA |
| | | glycans | | | |
| | β-haptoglobin | ↑ α2-3Neu5Ac | MAA | [22] | anti-α2-3- |
| | | | | | linked sialic |
| | | | | | acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | α-1-antitrypsin | ↑ tetra- | CE | [20] | DBA, PHA- |
| | | antennary | | | E, PHA-L |
| | | glycans | | | |
| | α-1-antitrypsin | ↑ Le^{x} | CE | [20] | Antibody |
| | | | | | against Le^{x}, |
| | | | | | LTA |
| | α-1-antitrypsin | ↓ tri-, tetra- | LTA affinity | [22] | DBA, PHA- |
| | | antennary | separation AND | | E, PHA-L |
| | | glycans | PAGE | | |
| | α-1-antitrypsin | ↓ α2-3Neu5Ac | LTA affinity | [22] | MAA, anti- |
| | | | separation AND | | α2-3-linked |
| | | | PAGE | | sialic acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | α-1-antitrypsin | ↑ α2-6Neu5Ac | LTA affinity | [22] | SNA, TJA-I |
| | | | separation AND | | |
| | | | PAGE | | |
| | α-1-antitrypsin | ↑ core fucose | LTA affinity | [22] | AOL, |
| | | | separation AND | | PhoSL |
| | | | PAGE | | |
| | α-1-antitrypsin | ↑ bi-antennary | Con A | [22] | NPA, GNA |
| | | glycans | | | |
| | α-1-antitrypsin | ↑ α2-6Neu5Ac | SNA | [22] | TJA-I, SCA |
| | α-1-antitrypsin | ↓α2-3Neu5Ac | MAA | [22] | anti-α2-3- |
| | | | | | linked sialic |
| | | | | | acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | α-1- | ↑ tetra- | CE | [20] | DBA, PHA- |
| | antichymotrypsin | antennary | | | E, PHA-L |
| | | glycans | | | |
| | α-1- | ↑ Le^{x} | CE | [20] | Antibody |
| | antichymotrypsin | | | | against Le^{x}, |
| | | | | | LTA |
| | α-1- | ↑ sialyl Le^{x} | 2D PAGE and LC | [21] | Antibody |
| | antichymotrypsin | | | | against |
| | | | | | sLe^{x}, SNA, |
| | | | | | TJA-I, MAA, |
| | | | | | anti-α2-3- |
| | | | | | linked sialic |
| | | | | | acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | α-1- | ↑ α2-6Neu5Ac | 2D PAGE and LC | [21] | SNA, TJA-I |
| | antichymotrypsin | | | | |
| | α-1- | ↓ α2-3Neu5Ac | 2D PAGE and LC | [21] | MAA, anti- |
| | antichymotrypsin | | | | α2-3-linked |
| | | | | | sialic acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | transferrin | ↓ tri-antennary | CE | [20] | DBA, PHA- |
| | | glycans | | | E, PHA-L |
| | hemopexin | ↑ Le^{x} | CE | [20] | Antibody |
| | | | | | against Le^{x}, |
| | | | | | LTA |
| | lgG | ↓ galactose | 2D PAGE and LC | [21] | RCA, |
| | | | | | RCA120, |
| | | | | | ABA, |
| | | | | | Jacalin |
| | | | | | (DSA), |
| | | | | | AlloA, ECL, |
| | | | | | PNA |
| | lgG | ↓ sialic acid | 2D PAGE and LC | [21] | SNA, TJA-I, |
| | | | | | MAA, anti- |
| | | | | | α2-3-linked |
| | | | | | sialic acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | CA125 (MUC16) | ↑ sialyl Tn | VVA lectin after | [23] | SNA, TJA-I, |
| | | antigen | sialidase detection | | MAA, anti- |
| | | | by | | α2-3-linked |
| | | | | | sialic acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | CA125 (MUC16) | ↑ sialyl T | anticarbohydrate | [23] | SNA, TJA-I, |
| | | antigen | IgM antibodies 3C9 | | MAA, anti- |
| | | | after sialidase | | α2-3-linked |
| | | | detection | | sialic acid |
| | | | | | antibody |
| | | | | | (i.e. |
| | | | | | HYB4i.e. |
| | | | | | i.e. HYB4), , |
| | | | | | Siglec 1, |
| | | | | | Siglec 4 or |
| | | | | | Siglec 8 |
| | CA15-3 (MUC1) | ↑ sialyl Tn | VVA lectin after | [23] | SNA, TJA-I, |
| | | antigen | sialidase detection | | MAA, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | CA15-3 (MUC1) | ↑ core fucose | PAGE/LC | [24] | PhoSL, |
| | | | | | AOL |
| | CA15-3 (MUC1) | ↑ bi-antennary | PAGE/LC | [24] | Con A |
| | | glycans | | | |
| | CA15-3 (MUC1) | ↓ tri-, tetra- | PAGE/LC | [24] | PHA-E, |
| | | antennary | | | PHA-L, |
| | | glycans | | | DBA |
| | CA15-3 (MUC1) | ↑ antennary | PAGE/LC | [24] | AAL, TJA II, |
| | | fucose | | | UEA-I, |
| | | | | | LCA, PSL, |
| | | | | | AAA, LTA |
| | human epididymis | ↑ Le^{y} antigen | Antibody against | [25] | UEA-I |
| | protein 4 (HE4) | | Lewis^{y} glycan | | |
| | Clusterin | ↑ α2-6Neu5Ac | SNA | [26] | TJA-I, SCA |
| | leucine-rich α-2- | ↑ α2-6Neu5Ac | SNA | [26] | TJA-I, SCA |
| | glycoprotein | | | | |
| Breast | CA15-3 (MUC1) | ↑ sulfated | Galectin 4 | [27] | SBA, ABA, |
| | | core1 glycan | | | VVA, |
| | | | | | Jacalin |
| | | | | | (DSA), |
| | | | | | BPL, PNA, |
| | | | | | GSL1, SJA |
| | CA15-3 (MUC1) | ↑ Tn, sialyl Tn | | [28] | SBA, DBA, |
| | | antigens | | | VVA, SNA, |
| | | | | | SNA, TJA-I, |
| | | | | | MAA, anti- |
| | | | | | α2-3-linked |
| | | | | | sialic acid |
| | | | | | antibody |
| | | | | | (i.e. HYB4) |
| | CA15-3 (MUC1) | change sialyl | LC | [29] | SNA, TJA-I, |
| | | T, Tn antigens | | | MAA, anti- |
| | | | | | α2-3-linked |
| | | | | | sialic acid |
| | | | | | antibody |
| | | | | | (i.e. |
| | | | | | HYB4), |
| | | | | | Siglec 1, |
| | | | | | Siglec 4 or |
| | | | | | Siglec 8; |
| | | | | | SBA, ABA |
| | | | | | VVA, BPL, |
| | | | | | Jacalin, |
| | | | | | PNA |
| | CA15-3 (MUC1) | change α2- | LC | [29] | antibody |
| | | 8Neu5Ac | | | against |
| | | | | | poly(sialic |
| | | | | | acid), |
| | | | | | Siglec 7 or |
| | | | | | Siglec 11 |
| | CA15-3 (MUC1) | change in | LC | [29] | SNA, TJA-I, |
| | | sialylation | | | MAA, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | CA15-3 (MUC1) | change in core | LC | [29] | RCA, |
| | | 2 glycan | | | RCA120, |
| | | | | | ABA, |
| | | | | | Jacalin |
| | | | | | (DSA), |
| | | | | | PNA, WGA |
| | CA15-3 | change in | MAA | [30] | SNA, TJA-I, |
| | | sialylation | | | MAA, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | CA15-3 (MUC1) | change in | MAA, SNA, | [30] | SNA, TJA-I, |
| | | sialylation | TVA = WGA | | MAA, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | CA27.29 | change in | MAA | [30] | SNA, TJA-I, |
| | | sialylation | | | MAA, anti- |
| | | | | | α2-3-linked |
| | | | | | sialic acid |
| | | | | | antibody |
| | | | | | (i.e. |
| | | | | | HYB4), , |
| | | | | | Siglec 1, |
| | | | | | Siglec 4 or |
| | | | | | Siglec 8 |
| | HER2 | change in | UEA | [30] | TJA II, AAL, |
| | | antennary | | | LCA, PSL, |
| | | fucose | | | AAA, LTA |
| | HER2 | change in | MAA, SNA, | [30] | SNA, TJA-I, |
| | | sialylation | TVA = WGA | | MAA, anti- |
| | | | | | α2-3-linked |
| | | | | | sialic acid |
| | | | | | antibody |
| | | | | | (i.e. |
| | | | | | HYB4), , |
| | | | | | Siglec 1, |
| | | | | | Siglec 4 or |
| | | | | | Siglec 8 |
| | CEA | change in tri-, | | [31] | PHA-E, |
| | | tetra- | | | PHA-L, |
| | | antennary | | | DBA |
| | | glycans | | | |
| Colorectal | β-haptoglobin | ↑ antennary | AAL | [32] | TJA II, |
| | | fucose | | | UEA-I, |
| | | | | | LCA, PSL, |
| | | | | | AAA, LTA |
| | β-haptoglobin | ↑ antennary | AAL | [33] | TJA II, |
| | | fucose | | | UEA-I, |
| | | | | | LCA, PSL, |
| | | | | | AAA, LTA |
| | β-haptoglobin | ↑ bi-antennary | PHA-E | [32] | Con A, |
| | | glycans | | | PHA-L, |
| | | | | | DBA |
| | β-haptoglobin | ↑ | AAL, AOL, LTA | [34] | TJA II, |
| | | antennary/core | | | UEA-I, |
| | | fucose | | | LCA, PSL, |
| | | | | | AAA, |
| | | | | | PhoSL |
| | β-haptoglobin | ↑ dimer: Leª | mouse monoclonal | [35] | |
| | | on Leª | antibody NCC-ST- | | |
| | | | 421, | | |
| | β-haptoglobin | ↑ Galβ1- | Galectin 3 | [36] | ECA, AlloA |
| | | 4GIcNAc | | | |
| | Carcinoembryonic | ↑ Le^{x} | LTA, Antibody | [37] | |
| | antigen (CEA) | | against sialyl | | |
| | | | Lewis^{x} glycan | | |
| | CEA | ↑ Le^{y} | UEA-I, Antibody | [37] | |
| | | | against sialyl | | |
| | | | Lewis^{y} glycan | | |
| | CEA | ↑ α2-3Neu5Ac | MAA | [37] | anti-α2-3- |
| | | | | | linked sialic |
| | | | | | acid |
| | | | | | antibody |
| | | | | | (i.e. HYB4), |
| | | | | | Siglec 1, |
| | | | | | Siglec 4 or |
| | | | | | Siglec 8 |
| | CEA | ↑ α-D-Man | NPA | [37] | Con A, |
| | | | | | GNA |
| | CEA | ↑ tri-, tetra- | PHA-L | [37] | PHA-E, |
| | | antennary | | | DBA |
| | | glycans | | | |
| | CEA | ↑ mannose, | DC-SIGN | [37] | NPA, Con |
| | | fucose | | | A, GNA, |
| | | | | | AAL, TJA II, |
| | | | | | UEA-I, |
| | | | | | LCA, PSL, |
| | | | | | AAA, LTA, |
| | | | | | AOL, |
| | | | | | PhoSL |
| | CEA | ↓ terminal | MGBL | [37] | DBA, SBA, |
| | | GalNAc | | | VVA, HPA, |
| | | | | | WFA |
| | CEA | ↑ Gal · 1- | Galectin 3 | | |
| | | 4GlcNAc | | | |
| | CA 19-9 (MUC1) | ↑ T antigen | SBA | [37] | ABA |
| | CA 19-9 (MUC1) | ↑ Galβ1- | PNA | [37] | ABA, |
| | | 3GalNAc | | | Jacalin |
| | CA 19-9 (MUC1) | ↑ antennary | UEA | [37] | TJA II, AAL, |
| | | fucose | | | LCA, PSL, |
| | | | | | AAA, LTA |
| | CA 19-9 (MUC1) | ↑ α2-3Neu5Ac | MAA | [37] | anti-α2-3- |
| | | | | | linked sialic |
| | | | | | acid |
| | | | | | antibody |
| | | | | | (i.e. HYB4), |
| | | | | | Siglec 1, |
| | | | | | Siglec 4 or |
| | | | | | Siglec 8 |
| | CA 19-9 (MUC1) | ↑ α2-6Neu5Ac | SNA | [37] | TJA-I |
| | CA 19-9 (MUC1) | ↓ tri-, tetra- | PHA-E, PHA-L | [37] | DBA |
| | | antennary | | | |
| | | glycans | | | |
| | CA 19-9 (MUC1) | ↑ terminal | MGBL | [37] | DBA, SBA, |
| | | GalNAc | | | HPA, WFA |
| | Complement C3 | ↑ antennary | AAL | [38] | TJA II, |
| | (UniProtKB: | fucose | | | UEA-I, |
| | P01024) | | | | LCA, PSL, |
| | | | | | AAA, LTA |
| | Complement C3 | ↑ Gal β 1- | PNA | [38] | ABA, |
| | (UniProtKB: | 3GalNAc | | | Jacalin |
| | P01024) | | | | |
| | Complement C3 | ↑ α2-3Neu5Ac | MAA | [38] | anti-α2-3- |
| | (UniProtKB: | | | | linked sialic |
| | P01024) | | | | acid |
| | | | | | antibody |
| | | | | | (i.e. HYB4), |
| | | | | | Siglec 1, |
| | | | | | Siglec 4 or |
| | | | | | Siglec 8 |
| | Complement C3 | ↑ α2-6Neu5Ac | SNA | [38] | TJA-I |
| | (UniProtKB: | | | | |
| | P01024) | | | | |
| | Kininogen-I | ↑ high | Con A | [38] | NPA, GNA |
| | (UniProtKB: | mannose | | | |
| | P01042) | | | | |
| | Kininogen-I | ↑ antennary | AAL | [38] | TJA II, |
| | (UniProtKB: | fucose | | | UEA-I, |
| | P01042) | | | | LCA, PSL, |
| | | | | | AAA, LTA |
| | Kininogen-I | ↑ Gal β 1- | PNA | [38] | ABA, |
| | (UniProtKB: | 3GalNAc | | | Jacalin |
| | P01042) | | | | |
| | Kininogen-I | ↑ α2-3Neu5Ac | MAA | [38] | anti-α2-3- |
| | (UniProtKB: | | | | linked sialic |
| | P01042) | | | | acid |
| | | | | | antibody |
| | | | | | (i.e. HYB4), |
| | | | | | Siglec 1, |
| | | | | | Siglec 4 or |
| | | | | | Siglec 8 |
| | Kininogen- | ↑ α2-6Neu5Ac | SNA | [38] | TJA-I |
| | I(UniProtKB: | | | | |
| | P01042) | | | | |
| | Histidine-rich | ↑ antennary | AAL | [38] | TJA II, |
| | glycoprotein | fucose | | | UEA-I, |
| | (UniProtKB: | | | | LCA, PSL, |
| | P04196) | | | | AAA, LTA |
| | Histidine-rich | ↑ α2-6Neu5Ac | SNA | [38] | TJA-I |
| | glycoprotein | | | | |
| | (UniProtKB: | | | | |
| | P04196) | | | | |
| Pancreatic | α₁-β-glycoprotein | ↑ Neu5Ac | SNA | [39] | TJA-I, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | Amyloid p- | ↑ Neu5Ac | SNA | [39] | TJA-I, anti- |
| | component | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | β-2-glycoprotein 1 | ↑ antennary | AAL | [40] | TJA II, |
| | (P02749) | fucose | | | UEA-I, |
| | | | | | LCA, PSL, |
| | | | | | AAA, LTA |
| | β-2-glycoprotein 1 | 1 α2-3Neu5Ac | MAA | [40] | anti-α2-3- |
| | (UniProtKB: | | | | linked sialic |
| | P02749) | | | | acid |
| | | | | | antibody |
| | | | | | (i.e. HYB4), |
| | | | | | Siglec 1, |
| | | | | | Siglec 4 or |
| | | | | | Siglec 8 |
| | β-2-glycoprotein 1 | ↑ α2-6Neu5Ac | SNA | [40] | TJA-I |
| | (UniProtKB: | | | | |
| | P02749) | | | | |
| | β-2-glycoprotein 1 | ↑ high | Con A | [40] | NPA, GNA |
| | (UniProtKB: | mannose | | | |
| | P02749) | | | | |
| | β-2-glycoprotein 1 | ↑ Gal β 1- | PNA | [40] | ABA, |
| | (UniProtKB: | 3GalNAc | | | Jacalin |
| | P02749) | | | | |
| | hemopexin | ↑ antennary | AAL | [40] | TJA II, |
| | (UniProtKB: | fucose | | | UEA-I, |
| | P02790) | | | | LCA, PSL, |
| | | | | | AAA, LTA |
| | hemopexin | ↑ α2-3Neu5Ac | MAA | [40] | anti-α2-3- |
| | (UniProtKB: | | | | linked sialic |
| | P02790) | | | | acid |
| | | | | | antibody |
| | | | | | (i.e. HYB4), |
| | | | | | Siglec 1, |
| | | | | | Siglec 4 or |
| | | | | | Siglec 8 |
| | hemopexin | ↑ α2-6Neu5Ac | SNA | [40] | TJA-I |
| | (UniProtKB: | | | | |
| | P02790) | | | | |
| | hemopexin | ↑ high | Con A | [40] | NPA, GNA |
| | (UniProtKB: | mannose | | | |
| | P02790) | | | | |
| | haptoglobin-related | ↑ antennary | AAL | [40] | TJA II, |
| | protein (UniProtKB: | fucose | | | UEA-I, |
| | P00739) | | | | LCA, PSL, |
| | | | | | AAA, LTA |
| | haptoglobin-related | ↑ α2-3Neu5Ac | MAA | [40] | anti-α2-3- |
| | protein (UniProtKB: | | | | linked sialic |
| | P00739) | | | | acid |
| | | | | | antibody |
| | | | | | (i.e. HYB4), |
| | | | | | Siglec 1, |
| | | | | | Siglec 4 or |
| | | | | | Siglec 8 |
| | haptoglobin-related | ↑ α2-6Neu5Ac | SNA | [40] | TJA-I |
| | protein (UniProtKB: | | | | |
| | P00739) | | | | |
| | haptoglobin-related | ↑ high | Con A | [40] | NPA, GNA |
| | protein (UniProtKB: | mannose | | | |
| | P00739) | | | | |
| | haptoglobin-related | ↑ Gal β 1- | PNA | [40] | ABA, |
| | protein (UniProtKB: | 3GalNAc | | | Jacalin |
| | P00739) | | | | |
| | serum amyloid P- | ↑ antennary | AAL | [40] | TJA II, |
| | component | fucose | | | UEA-I, |
| | (UniProtKB: | | | | LCA, PSL, |
| | P02743) | | | | AAA, LTA |
| | serum amyloid P- | ↑ α2-3Neu5Ac | MAA | [40] | anti-α2-3- |
| | component | | | | linked sialic |
| | (UniProtKB: | | | | acid |
| | P02743) | | | | antibody |
| | | | | | (i.e. HYB4), |
| | | | | | Siglec 1, |
| | | | | | Siglec 4 or |
| | | | | | Siglec 8 |
| | serum amyloid P- | ↑ α2-6Neu5Ac | SNA | [40] | TJA-I |
| | component | | | | |
| | (UniProtKB: | | | | |
| | P02743) | | | | |
| | serum amyloid P- | ↑ high | Con A | [40] | NPA, GNA |
| | component | mannose | | | |
| | (UniProtKB: | | | | |
| | P02743) | | | | |
| | serum amyloid P- | ↑ Gal β 1- | PNA | [40] | ABA, |
| | component | 3GalNAc | | | Jacalin |
| | (UniProtKB: | | | | (DSA) |
| | P02743) | | | | |
| | clusterin | ↑ antennary | AAL | [40] | TJA II, |
| | (UniProtKB: | fucose | | | UEA-I, |
| | P10909) | | | | LCA, PSL, |
| | | | | | AAA, LTA |
| | clusterin | ↑ α2-3Neu5Ac | MAA | [40] | anti-α2-3- |
| | (UniProtKB: | | | | linked sialic |
| | P10909) | | | | acid |
| | | | | | antibody |
| | | | | | (i.e. HYB4), |
| | | | | | Siglec 1, |
| | | | | | Siglec 4 or |
| | | | | | Siglec 8 |
| | clusterin | ↑ α2-6Neu5Ac | SNA | [40] | TJA-I |
| | (UniProtKB: | | | | |
| | P10909) | | | | |
| | clusterin | ↑ Gal β 1- | PNA | [40] | ABA, |
| | (UniProtKB: | 3GalNAc | | | Jacalin |
| | P10909) | | | | |
| | antithrombin-III | ↑ antennary | AAL | [40] | TJA II, |
| | (UniProtKB: | fucose | | | UEA-I, |
| | P01008) | | | | LCA, PSL, |
| | | | | | AAA, LTA |
| | antithrombin-III | ↑ α2-3Neu5Ac | MAA | [40] | anti-α2-3- |
| | (UniProtKB: | | | | linked sialic |
| | P01008) | | | | acid |
| | | | | | antibody |
| | | | | | (i.e. HYB4), |
| | | | | | Siglec 1, |
| | | | | | Siglec 4 or |
| | | | | | Siglec 8 |
| | antithrombin-III | ↑ α2-6Neu5Ac | SNA | [40] | TJA-I |
| | (UniProtKB: | | | | |
| | P01008) | | | | |
| | antithrombin-III | ↑ high | Con A | [40] | NPA, GNA |
| | (UniProtKB: | mannose | | | |
| | P01008) | | | | |
| | antithrombin-III | ↑ Gal β 1- | PNA | [40] | ABA, |
| | (UniProtKB: | 3GalNAc | | | Jacalin |
| | P01008) | | | | (DSA) |
| | kininogen-1 | ↑ antennary | AAL | [40] | TJA II, |
| | (UniProtKB: | fucose | | | UEA-I, |
| | P01042) | | | | LCA, PSL, |
| | | | | | AAA, LTA |
| | kininogen-1 | ↑ α2-3Neu5Ac | MAA | [40] | anti-α2-3- |
| | (UniProtKB: | | | | linked sialic |
| | P01042) | | | | acid |
| | | | | | antibody |
| | | | | | (i.e. HYB4), |
| | | | | | Siglec 1, |
| | | | | | Siglec 4 or |
| | | | | | Siglec 8 |
| | kininogen-1 | ↑ α2-6Neu5Ac | SNA | [40] | TJA-I |
| | (UniProtKB: | | | | |
| | P01042) | | | | |
| | kininogen-1 | ↑ high | Con A | [40] | NPA, GNA |
| | (UniProtKB: | mannose | | | |
| | P01042) | | | | |
| | kininogen-1 | ↑ Gal β 1- | PNA | [40] | ABA, |
| | (UniProtKB: | 3GalNAc | | | Jacalin |
| | P01042) | | | | (DSA) |
| | plasma protease | ↑ α2-6Neu5Ac | SNA | [40] | TJA-I |
| | C1 inhibitor | | | | |
| | (UniProtKB: | | | | |
| | P05155) | | | | |
| | β-haptoglobin | ↑ antennary | AAL | [41] | TJA II, |
| | | fucose | | | UEA-I, |
| | | | | | LCA, PSL, |
| | | | | | AAA, LTA |
| | β-haptoglobin | ↑ antennary | AAL | [42] | TJA II, |
| | | fucose | | | UEA-I, |
| | | | | | LCA, PSL, |
| | | | | | AAA, LTA |
| | β-haptoglobin | ↑ core fucose | AOL | [41] | PhoSL |
| | β-haptoglobin | ↑ core fucose | PhoSL | [43] | AOL |
| | α-1- | ↑ antennary | AAL | [42] | TJA II, |
| | antichymotrypsin | fucose | | | UEA-I, |
| | | | | | LCA, PSL, |
| | | | | | AAA, LTA |
| | thrombospondin-1 | ↑ antennary | AAL | [42] | TJA II, |
| | | fucose | | | UEA-I, |
| | | | | | LCA, PSL, |
| | | | | | AAA, LTA |
| | α-1-antitrypsin | ↑ antennary | AAL | [42] | TJA II, |
| | | fucose | | | UEA-I, |
| | | | | | LCA, PSL, |
| | | | | | AAA, LTA |
| | Mucin (CAM 17.1) | ↑ β-D- | WGA | [44, | DSA, LEL, |
| | | GlcNAc, | | 45] | SNA, TJA-I |
| | | Neu5Ac | | | |
| | MUC16 | ↑ antennary | AAL | [46, | TJA II, |
| | | fucose | | 47] | UEA-I, |
| | | | | | LCA, PSL, |
| | | | | | AAA, LTA |
| | MUC16 | ↓ T antigen | BPL, Jacalin | [46] | SBA, VVA, |
| | | | (DSA), PNA | | ABA, GSL1, |
| | | | | | SJA |
| | MUC16 | ↓ Gal-GlcNAc | ECL, PHA-L | [46] | PHA-E, |
| | | | | | AlloA, ECA, |
| | MUC16 | ↓ GalNAc | DBA, GSL1, SBA, | [46] | ABA, BPL, |
| | | | VVL, SJA | | PNA |
| | MUC16 | ↓ GlcNAc | GSL2, STL | [46] | DSA, LEL, |
| | | | | | WGA |
| | MUC16 | ↓ mannose | Con A | [46] | GNA, NPA |
| | MUC5ac | ↑ T antigen | Jacalin | [46] | SBA, ABA, |
| | | | | | VVA, BPL, |
| | | | | | PNA |
| | MUC5ac | ↑ antennary | AAL | [46] | TJA II, EA-I, |
| | | fucose | | | LCA, PSL, |
| | | | | | AAA, LTA |
| | MUC5ac | ↑ T antigen | Jacalin (DSA) | [46] | SBA, ABA, |
| | | | | | VVA, BPL, |
| | | | | | PNA, |
| | | | | | GSL1, SJA |
| | MUC5ac | ↓ Gal-GlcNAc | ECA, PHA-L, | [46] | PHA-E, |
| | | | RCA120 | | RCA |
| | MUC5ac | ↓ GalNAc | DBA, VVA, SJA | [46] | GSL1, SBA, |
| | | | | | ABA, BPL, |
| | | | | | PNA |
| | MUC5ac | ↓ GlcNAc | GSL 2, LEL, STL | [46] | DSA, LEL, |
| | | | | | WGA, |
| | | | | | GSL2, STL |
| | MUC1 | ↓ Gal-GlcNAc, | PHA-L | [46] | ECA, PHA- |
| | | tetra- | | | L, RCA120, |
| | | antennary | | | PHA-E, |
| | | glycans | | | RCA; DBA |
| | MUC1 | ↓T antigen | Jacalin (DSA) | [46] | SBA, ABA, |
| | | | | | VVA, BPL, |
| | | | | | PNA, |
| | | | | | GSL1, SJA |
| | MUC1 | ↓ GalNAc | DBA | [46] | VVA, SJA, |
| | | | | | GSL1, SBA, |
| | | | | | ABA, BPL, |
| | | | | | PNA |
| | MUC1 | 1 Gal α 1-3Gal | GSL 1 | [46] | |
| | MUC1 | ↓ GlcNAc | GSL 2, LEL, STL | [46] | DSA, LEL, |
| | | | | | WGA, |
| | | | | | GSL2, STL |
| Thyroid | Thyroglobulin (TG) | ↓ antennary | LCA | [48, | TJA II, AAL, |
| | | fucose | | 49] | UEA-I, PSL, |
| | | | | | AAA, LTA |
| | TG | ↑ terminal | RCA | [50] | RCA120, |
| | | galactose | | | ABA, AlloA, |
| | | | | | Jacalin |
| | | | | | (DSA), |
| | | | | | ECL, PNA |
| | TG | ↑ Gal-GlcNAc | LC assays | [50] | ECA, PHA- |
| | | | | | L, RCA120, |
| | | | | | PHA-E, |
| | | | | | RCA |
| | TG | ↑ tri-antennary | LC assays | [50] | PHA-E, |
| | | glycans | | | PHA-L, |
| | | | | | DBA |
| | TG | ↑ antennary | LC assays | [50] | TJA II, AAL, |
| | | fucose | | | UEA-I, |
| | | | | | LCA, PSL, |
| | | | | | AAA, LTA |
| | TG | ↑ mannose | LC assays | [50] | Con A, |
| | | | | | NPA, GNA |
| Liver | α₁-antitrypsin | ↑ antennary | LCA | [51] | TJA II, |
| | (AAT) | fucose | | | UEA-I, AAL, |
| | | | | | PSL, AAA, |
| | | | | | LTA |
| | α₁-antitrypsin | ↑ antennary | AAL | [52, | TJA II, |
| | (AAT) | fucose | | 53] | UEA-I, |
| | | | | | LCA, PSL, |
| | | | | | AAA, LTA |
| | α-fetoprotein (AFP) | ↑ antennary | LCA | [51, | TJA II, AAL, |
| | | fucose | | 54] | UEA-I, PSL, |
| | | | | | AAA, LTA |
| | α-fetoprotein (AFP) | ↑ antennary | AAL | [54] | TJA II, |
| | | fucose | | | UEA-I, |
| | | | | | LCA, PSL, |
| | | | | | AAA, LTA |
| | AFP-L3 | ↑ antennary | LCA | [55, | TJA II, |
| | | fucose | | 56] | UEA-I, PSL, |
| | | | | | AAA, LTA |
| | transferrin | ↑ antennary | LCA | [51] | TJA II, |
| | | fucose | | | UEA-I, PSL, |
| | | | | | AAA, LTA |
| | α₁- | ↑ antennary | AAL | [52] | TJA II, |
| | antichymotrypsin | fucose | | | UEA-I, |
| | (AAT) | | | | LCA, PSL, |
| | | | | | AAA, LTA |
| | α-1-acid | ↑ antennary | AAL | [52] | TJA II, |
| | glycoprotein 1 | fucose | | | UEA-I, |
| | | | | | LCA, PSL, |
| | | | | | AAA, LTA |
| | ceruloplasmin | ↑ antennary | AAL | [52] | TJA II, |
| | | fucose | | | UEA-I, |
| | | | | | LCA, PSL, |
| | | | | | AAA, LTA |
| | α-2-macroglobulin | ↑ antennary | AAL, LCA | [54] | TJA II, |
| | | fucose | | | UEA-I, PSL, |
| | | | | | AAA, LTA |
| | α-2-HS- | ↑ antennary | AAL | [53] | TJA II, |
| | glycoprotein | fucose | | | UEA-I, |
| | | | | | LCA, PSL, |
| | | | | | AAA, LTA |
| | Fetuin A | ↑ antennary | AAL | [57] | TJA II, |
| | | fucose | | | UEA-I, |
| | | | | | LCA, PSL, |
| | | | | | AAA, LTA |
| | hemopexin | ↑ antennary | AAL | [54, | TJA II, |
| | | fucose | | 57] | UEA-I, |
| | | | | | LCA, PSL, |
| | | | | | AAA, LTA |
| | hemopexin | ↑ antennary | LCA | [54] | TJA II, AAL, |
| | | fucose | | | UEA-I, PSL, |
| | | | | | AAA, LTA |
| | Ceruloplasmin | ↑ antennary | AAL, LCA | [58] | TJA II, |
| | | fucose | | | UEA-I, PSL, |
| | | | | | AAA, LTA |
| | C3 complement | ↑ antennary | AAL, LCA | [58] | TJA II, |
| | | fucose | | | UEA-I, PSL, |
| | | | | | AAA, LTA |
| | Histidine rich | ↑ antennary | AAL, LCA | [58] | TJA II, |
| | glycoprotein | fucose | | | UEA-I, PSL, |
| | | | | | AAA, LTA |
| | Monocyte | ↑ antennary | AAL, LCA | [58] | TJA II, |
| | differentiation | fucose | | | UEA-I, PSL, |
| | antigen CD14 | | | | AAA, LTA |
| | Hepatocyte growth | ↑ antennary | AAL, LCA | [58] | TJA II, |
| | factor activator | fucose | | | UEA-I, PSL, |
| | | | | | AAA, LTA |
| Lung | β-haptoglobin | ↑ antennary | AAL | [59] | TJA II, |
| | | fucose | | | UEA-I, PSL, |
| | | | | | AAA, LCA, |
| | | | | | LTA |
| | β-haptoglobin | ↑ antennary | AAL | [59] | TJA II, |
| | | fucose | | | UEA-I, PSL, |
| | | | | | AAA, LCA, |
| | | | | | LTA |
| | β-haptoglobin | ↑ antennary | MS | [60] | AAL, TJA II, |
| | | fucose | | | UEA-I, |
| | | | | | LCA, PSL, |
| | | | | | AAA, LTA |
| | β-haptoglobin | ↑ core fucose | MS | [60] | AOL, |
| | | | | | PhoSL |
| | β-haptoglobin | ↑ tri-, tetra- | MS | [60] | PHA-E, |
| | | antennary | | | PHA-L, |
| | | glycans | | | DBA |
| | β-haptoglobin | ↑ α2-6Neu5Ac | MS | [61] | SNA, TJA-I |
| | β-haptoglobin | ↑ antennary | MS | [61] | AAL, TJA II, |
| | | fucose | | | UEA-I, |
| | | | | | LCA, PSL, |
| | | | | | AAA, LTA |
| | β-haptoglobin | ↑ sialyl Le^{x} | LC | [62] | SNA, TJA-I, |
| | | | | | MAA, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | β-haptoglobin | ↑ tri-antennary | LC | [62] | PHA-E, |
| | | | | | PHA-L, |
| | | | | | DBA |
| | β-haptoglobin | ↑ sialic acid | LC | [62] | SNA, TJA-I, |
| | | | | | MAA, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | fibronectin | ↑ Galβ1- | PNA | [63] | ABA, |
| | | 3GalNAc | | | Jacalin |
| | | | | | (DSA) |
| | α₁-acid | ↑ antennary | AAL | [64] | TJA II, |
| | glycoprotein | fucose | | | UEA-I, |
| | | | | | LCA, PSL, |
| | | | | | AAA, LTA |
| | α₁-acid | ↑ sialyl Le^{x} | Antibody against | [64] | SNA, TJA-I, |
| | glycoprotein | | sLe^{x} | | MAA, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | α-1-antitrypsin | ↑ antennary | AAL | [65] | TJA II, |
| | | fucose | | | UEA-I, |
| | | | | | LCA, PSL, |
| | | | | | AAA, LTA |
| | α-1-antitrypsin | ↑ β-Gal, | RCA120 | [65] | RCA, ECL, |
| | | Galβ1- | | | AlloA |
| | | 4GlcNAc | | | |
| | α-1-antitrypsin | 1 α-Gal and α- | BS-I | [65] | DBA, SBA, |
| | | GalNAc | | | HPA |
| | α-1-antitrypsin | ↑ (GlcNAc)ₙ | WGA | [65] | LEL |
| | α-1-antitrypsin | ↑ Branched | PWM | [65] | |
| | | (LacNAc)ₙ | | | |
| | α-1-antitrypsin | ↑ high- | GNA | [65] | Con A, NPA |
| | | mannose, | | | |
| | | Manα1-3Man | | | |
| Stomach | α₁-acid | ↑ bi-antennary | Con A | [66] | NPA, GNA |
| | glycoprotein | glycans | | | |
| | α₁-acid | ↓ galactose | | [66] | RCA, |
| | glycoprotein | | | | RCA120, |
| | | | | | ABA, AlloA, |
| | | | | | Jacalin |
| | | | | | (DSA), |
| | | | | | ECL, PNA |
| | α₁-acid | ↑ Le^{x} | | [66] | LTA |
| | glycoprotein | | | | |
| | β-haptoglobin | ↑ sialyl Le^{x} | anti-sLe^{x} mouse | [67] | SNA, TJA-I, |
| | | (sLe^{x}) | monoclonal KM93 | | MAA, anti- |
| | | | antibody | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | β-haptoglobin | ↑ tri-, tetra- | LC/MS | [68] | PHA-E, |
| | | antennary | | | PHA-L, |
| | | glycans | | | DBA |
| | β-haptoglobin | ↑ antennary | LC/MS | [68] | AAL, TJA II, |
| | | fucose | | | UEA-I, |
| | | | | | LCA, PSL, |
| | | | | | AAA, LTA |
| | β-haptoglobin | ↑ sialyl-Leª | LC/MS | [68] | Antibody |
| | | (sLeª) | | | against |
| | | | | | sLeª, SNA, |
| | | | | | TJA-I, MAA, |
| | | | | | anti-α2-3- |
| | | | | | linked sialic |
| | | | | | acid |
| | | | | | antibody |
| | | | | | (i.e. HYB4), |
| | | | | | Siglec 1, |
| | | | | | Siglec 4 or |
| | | | | | Siglec 8 |
| | β-haptoglobin | ↑ sialyl-Leª | LC/MS | [68] | Antibody |
| | | (sLeª) | | | against |
| | | | | | sLeª, SNA, |
| | | | | | TJA-I, MAA, |
| | | | | | anti-α2-3- |
| | | | | | linked sialic |
| | | | | | acid |
| | | | | | antibody |
| | | | | | (i.e. HYB4) |
| | β-haptoglobin | ↑ antennary | AAL | [68] | TJA II, |
| | | fucose | | | UEA-I, |
| | | | | | LCA, PSL, |
| | | | | | AAA, LTA |
| | β-haptoglobin | ↑ (GlcNAc)ₙ | WGA | [68] | LEL |
| | β-haptoglobin | ↓ high | Con A | [68] | NPA, GNA |
| | | mannose | | | |
| | $\frac{leucine - rich - α2}{glycoprotein}$ | ↑ sialyl Le^{x} (sLe^{x}) | anti-sLe^{x} mouse monoclonal KM93 antibody | [67] | Antibody against sLeª, SNA, |
| | | | | | TJA-I, MAA, |
| | | | | | anti-α2-3- |
| | | | | | linked sialic |
| | | | | | acid |
| | | | | | antibody |
| | | | | | (i.e. HYB4), |
| | | | | | Siglec 1, |
| | | | | | Siglec 4 or |
| | | | | | Siglec 8 |
| Testicular | Human chorionic | ↑ fucose | LC-MS | [69] | AAL, TJA II, |
| | gonadotropin-β | | | | UEA-I, |
| | | | | | LCA, PSL, |
| | | | | | AAA, LTA, |
| | | | | | PhoSL, |
| | | | | | AOL |
| | Human chorionic | ↑ tri-antennary | LC-MS | [69] | PHA-E, |
| | gonadotropin-β | glycans | | | PHA-L, |
| | | | | | DBA |
| | AFP-L3 | ↑ antennary | LCA | [70] | AAL, TJA II, |
| | | fucose | | | UEA-I, |
| | | | | | LCA, PSL, |
| | | | | | AAA, LTA |
| Bladder | MUC1 | ↑ antennary | AAL | [71, | TJA II, |
| | | fucose | | 72] | UEA-I, |
| | | | | | LCA, PSL, |
| | | | | | AAA, LTA |
| | endoplasmin | ↑ antennary | AAL | [71, | TJA II, |
| | (HSP90B1) | fucose | | 72] | UEA-I, |
| | | | | | LCA, PSL, |
| | | | | | AAA, LTA |
| | Golgi apparatus | ↑ antennary | AAL | [71, | TJA II, |
| | protein 1 (GLG1) | fucose | | 72] | UEA-I, |
| | | | | | LCA, PSL, |
| | | | | | AAA, LTA |
| | prostatic acid | ↑ antennary | AAL | [71, | TJA II, |
| | phosphatase | fucose | | 72] | UEA-I, |
| | (ACPP) | | | | LCA, PSL, |
| | | | | | AAA, LTA |
| | lg gamma-2 chain | ↑ antennary | AAL | [71, | TJA II, |
| | C region (IGHG2) | fucose | | 72] | UEA-I, |
| | | | | | LCA, PSL, |
| | | | | | AAA, LTA |
| | deoxyribonuclease- | ↑ antennary | AAL | [71, | TJA II, |
| | 2-alpha | fucose | | 72] | UEA-I, |
| | (DNASE2A) | | | | LCA, PSL, |
| | | | | | AAA, LTA |
| | integrin | ↑ sialic acid | MS | [73] | SNA, TJA-I, |
| | | | | | MAA, anti- |
| | | | | | α2-3-linked |
| | | | | | sialic acid |
| | | | | | antibody |
| | | | | | (i.e. HYB4), |
| | | | | | Siglec 1, |
| | | | | | Siglec 4 or |
| | | | | | Siglec 8 |
| | integrin | ↑ tetra- | MS | [73] | PHA-E, |
| | | antennary | | | PHA-L, |
| | | glycans | | | DBA |
| | MUC16 | ↑ sialyl Tn | LC/MS | [74] | SNA, TJA-I, |
| | | | | | MAA, anti- |
| | | | | | α2-3-linked |
| | | | | | sialic acid |
| | | | | | antibody |
| | | | | | (i.e. HYB4) |
| | α-1-antitrypsin | ↑ high | Con A | [75] | NPA, GNA |
| | | mannose | | | |
| | α-1-antitrypsin | ↑ (GlcNAcβ1- | WGA | [75] | LEL |
| | | 4)ₙ | | | |

References as shown in Fehler! Verweisquelle konnte nicht gefunden werden. are as follows:
[1] C. Ohyama, M. Hosono, K. Nitta, M. Oh-eda, K. Yoshikawa, T. Habuchi, Y. Arai, M. Fukuda, Carbohydrate structure and differential binding of prostate specific antigen to Maackia amurensis lectin between prostate cancer and benign prostate hypertrophy, Glycobiology, 14 (2004) 671-679.
[2] K. Fukushima, T. Satoh, S. Baba, K. Yamashita, α1,2-Fucosylated and β-N-acetylgalactosaminylated prostate-specific antigen as an efficient marker of prostatic cancer, Glycobiology, 20 (2010) 452-460.
[3] T. Ishikawa, T. Yoneyama, Y. Tobisawa, S. Hatakeyama, T. Kurosawa, K. Nakamura, S. Narita, K. Mitsuzuka, W. Duivenvoorden, J. H. Pinthus, Y. Hashimoto, T. Koie, T. Habuchi, Y. Arai, C. Ohyama, An Automated Micro-Total Immunoassay System for Measuring Cancer-Associated 2,3-linked Sialyl N-Glycan-Carrying Prostate-Specific Antigen May Improve the Accuracy of Prostate Cancer Diagnosis, Int. J. Mol. Sci., 18 (2017) 15.
[4] D. Pihikova, P. Kasak, P. Kubanikova, R. Sokol, J. Tkac, Aberrant sialylation of a prostate-specific antigen: Electrochemical label-free glycoprofiling in prostate cancer serum samples, Anal. Chim. Acta, 934 (2016) 72-79.
[5] C. Ohyama, T. Koie, T. Yoneyama, Y. Tobisawa, Quantification of prostate cancer-associated aberrant glycosylation of prostate-specific antigen, Glycoscience: Biology and Medicine, Springer2015, pp. 1373-1377.
[6] E. Llop, M. Ferrer-Batalle, S. Barrabes, P. E. Guerrero, M. Ramirez, R. Saldova, P. M. Rudd, R. N. Aleixandre, J. Comet, R. de Llorens, R. Peracaula, Improvement of Prostate Cancer Diagnosis by Detecting PSA Glycosylation-Specific Changes, Theranostics, 6 (2016) 1190-1204.
[7] M. Ferrer-Batalle, E. Llop, M. Ramirez, R. N. Aleixandre, M. Saez, J. Comet, R. de Llorens, R. Peracaula, Comparative Study of Blood-Based Biomarkers, alpha 2,3-Sialic Acid PSA and PHI, for High-Risk Prostate Cancer Detection, International Journal of Molecular Sciences, 18 (2017) 12.
[8] T. Yoneyama, C. Ohyama, S. Hatakeyama, S. Narita, T. Habuchi, T. Koie, K. Mori, K. I. Hidari, M. Yamaguchi, T. Suzuki, Measurement of aberrant glycosylation of prostate specific antigen can improve specificity in early detection of prostate cancer, Biochem. Biophys. Res. Commun., 448 (2014) 390-396.
[9] N. Idil, I. Percin, V. Karakoc, H. Yavuz, N. Aksoz, A. Denizli, Concanavalin A immobilized magnetic poly (glycidyl methacrylate) beads for prostate specific antigen binding, Colloid Surf. B-Biointerfaces, 134 (2015) 461-468.
[10] M. V. Dwek, A. Jenks, A. J. Leathem, A sensitive assay to measure biomarker glycosylation demonstrates increased fucosylation of prostate specific antigen (PSA) in patients with prostate cancer compared with benign prostatic hyperplasia, Clin. Chim. Acta, 411 (2010) 1935-1939.

[11] K. Hagiwara, Y. Tobisawa, T. Kaya, T. Kaneko, S. Hatakeyama, K. Mori, Y. Hashimoto, T. Koie, Y. Suda, C. Ohyama, T. Yoneyama, Wisteria floribunda Agglutinin and Its Reactive-Glycan-Carrying Prostate-Specific Antigen as a Novel Diagnostic and Prognostic Marker of Prostate Cancer, Int. J. Mol. Sci., 18 (2017) 16.

[12] T. Kaya, T. Kaneko, S. Kojima, Y. Nakamura, Y. Ide, K. Ishida, Y. Suda, K. Yamashita, High-sensitivity immunoassay with surface plasmon field-enhanced fluorescence spectroscopy using a plastic sensor chip: Application to quantitative analysis of total prostate-specific antigen and GalNAcβ1-4GlcNAc-linked prostate-specific antigen for prostate cancer diagnosis, Anal. Chem., 87 (2015) 1797-1803.
[13] Q. K. Li, L. Chen, M. -H. Ao, J. H. Chiu, Z. Zhang, H. Zhang, D. W. Chan, Serum fucosylated prostate-specific antigen (PSA) improves the differentiation of aggressive from non-aggressive prostate cancers, Theranostics, 5 (2015) 267.
[14] K. Fujita, T. Hayashi, K. Matsuzaki, W. Nakata, M. Masuda, A. Kawashima, T. Ujike, A. Nagahara, M. Tsuchiya, Y. Kobayashi, S. Nojima, M. Uemura, E. Morii, E. Miyoshi, N. Nonomura, Decreased fucosylated PSA as a urinary marker for high Gleason score prostate cancer, Oncotarget, 7 (2016) 56643-56649.
[15] S. Takahashi, T. Sugiyama, M. Shimomura, Y. Kamada, K. Fujita, N. Nonomura, E. Miyoshi, M. Nakano, Site-specific and linkage analyses of fucosylated N-glycans on haptoglobin in sera of patients with various types of cancer: possible implication for the differential diagnosis of cancer, Glycoconjugate J., 33 (2016) 471-482.
[16] S. Kazuno, T. Fujimura, T. Arai, T. Ueno, K. Nagao, M. Fujime, K. Murayama, Multi-sequential surface plasmon resonance analysis of haptoglobin-lectin complex in sera of patients with malignant and benign prostate diseases, Anal. Biochem., 419 (2011) 241-249.
[17] S. -J. Yoon, S. -Y. Park, P. -C. Pang, J. Gallagher, J. E. Gottesman, A. Dell, J. -H. Kim, S. -I. Hakomori, N-glycosylation status of β-haptoglobin in sera of patients with prostate cancer vs. benign prostate diseases, Int. J. Oncol., 36 (2010) 193-203.
[18] T. Fujimura, Y. Shinohara, B. Tissot, P. C. Pang, M. Kurogochi, S. Saito, Y. Arai, M. Sadilek, K. Murayama, A. Dell, Glycosylation status of haptoglobin in sera of patients with prostate cancer vs. benign prostate disease or normal subjects, Int. J. Cancer, 122 (2008) 39-49.
[19] K. Fujita, M. Shimomura, M. Uemura, W. Nakata, M. Sato, A. Nagahara, Y. Nakai, S. Takamatsu, E. Miyoshi, N. Nonomura, Serum fucosylated haptoglobin as a novel prognostic biomarker predicting high-Gleason prostate cancer, The Prostate, 74 (2014) 1052-1058.
[20] S. Weiz, M. Wieczorek, C. Schwedler, M. Kaup, E. I. Braicu, J. Sehouli, R. Tauber, V. Blanchard, Acute-phase glycoprotein N-glycome of ovarian cancer patients analyzed by CE-LIF, Electrophoresis, 37 (2016) 1461-1467.
[21] R. Saldova, L. Royle, C. M. Radcliffe, U. M. Abd Hamid, R. Evans, J. N. Arnold, R. E. Banks, R. Hutson, D. J. Harvey, R. Antrobus, Ovarian cancer is associated with changes in glycosylation in both acute-phase proteins and IgG, Glycobiology, 17 (2007) 1344-1356.
[22] G. Turner, M. Goodarzi, S. Thompson, Glycosylation of alpha-1-proteinase inhibitor and haptoglobin in ovarian cancer: evidence for two different mechanisms, Glycoconjugate J., 12 (1995) 211-218.
[23] K. Chen, A. Gentry-Maharaj, M. Burnell, C. Steentoft, L. Marcos-Silva, U. Mandel, I. Jacobs, A. Dawnay, U. Menon, O. Blixt, Microarray Glycoprofiling of CA125 improves differential diagnosis of ovarian cancer, J. Proteome Res., 12 (2013) 1408-1418.
[24] R. Saldova, W. B. Struwe, K. Wynne, G. Elia, M. J. Duffy, P. M. Rudd, Exploring the glycosylation of serum CA125, International journal of molecular sciences, 14 (2013) 15636-15654.
[25] H. Zhuang, J. Gao, Z. Hu, J. Liu, D. Liu, B. Lin, Co-expression of Lewis y antigen with human epididymis protein 4 in ovarian epithelial carcinoma, PLOS One, 8 (2013) e68994.
[26] J. Wu, X. Xie, S. Nie, R. J. Buckanovich, D. M. Lubman, Altered expression of sialylated glycoproteins in ovarian cancer sera using lectin-based ELISA assay and quantitative glycoproteomics analysis, J. Proteome Res., 12 (2013) 3342-3352.
[27] H. Ideo, Y. Hinoda, K. Sakai, I. Hoshi, S. Yamamoto, M. Oka, K. Maeda, N. Maeda, S. Hazama, J. Amano, Expression of mucin 1 possessing a 3′-sulfated core1 in recurrent and metastatic breast cancer, Int. J. Cancer, 137 (2015) 1652-1660.
[28] S. A. Svarovsky, L. Joshi, Cancer glycan biomarkers and their detection-past, present and future, Anal. Methods, 6 (2014) 3918-3936.
[29] S. J. Storr, L. Royle, C. J. Chapman, U. M. A. Hamid, J. F. Robertson, A. Murray, R. A. Dwek, P. M. Rudd, The O-linked glycosylation of secretory/shed MUC1 from an advanced breast cancer patient's serum, Glycobiology, 18 (2008) 456-462.
[30] H. A. Badr, D. M. AlSadek, A. A. Darwish, A. I. ElSayed, B. O. Bekmanov, E. M. Khussainova, X. Zhang, W. C. Cho, L. B. Djansugurova, C. -Z. Li, Lectin approaches for glycoproteomics in FDA-approved cancer biomarkers, Expert review of proteomics, 11 (2014) 227-236.
[31] Y. Taeda, M. Nose, S. Hiraizumi, N. Ohuchi, Expression of L-PHA-binding proteins in breast cancer: Reconstitution and molecular characterization of beta 1-6 branched oligosaccharides in three-dimensional cell culture, Breast Cancer Res. Treat., 38 (1996) 313-324.
[32] S. Y. Park, S. J. Yoon, Y. T. Jeong, J. M. Kim, J. Y. Kim, B. Bernert, T. Ullman, S. H. Itzkowitz, J. H. Kim, S. i. Hakomori, N-glycosylation status of β-haptoglobin in sera of patients with colon cancer, chronic inflammatory diseases and normal subjects, Int. J. Cancer, 126 (2010) 142-155.
[33] Y. Takeda, S. Shinzaki, K. Okudo, K. Moriwaki, K. Murata, E. Miyoshi, Fucosylated haptoglobin is a novel type of cancer biomarker linked to the prognosis after an operation in colorectal cancer, Cancer, 118 (2012) 3036-3043.
[34] S. Y. Park, S. H. Lee, N. Kawasaki, S. Itoh, K. Kang, S. Hee Ryu, N. Hashii, J. M. Kim, J. Y. Kim, J. Hoe Kim, α1-¾ fucosylation at Asn 241 of β-haptoglobin is a novel marker for colon cancer: A combinatorial approach for development of glycan biomarkers, Int. J. Cancer, 130 (2012) 2366-2376.
[35] S. -Y. Park, S. -J. Yoon, S. -I. Hakomori, J. -M. Kim, J. -Y. Kim, B. Bernert, T. Ullman, S. H. Itzkowitz, J. H. Kim, Dimeric Lea (Lea-on-Lea) status of β-haptoglobin in sera of colon cancer, chronic inflammatory disease and normal subjects, Int. J. Oncol., 36 (2010) 1291-1297.
[36] R. S. Bresalier, J. C. Byrd, D. Tessler, J. Lebel, J. Koomen, D. Hawke, E. Half, K. F. Liu, N. Mazurek, C. Great Lakes-New England, A circulating ligand for galectin-3 glycoprotein elevated in individual is a haptoglobin-related with colon cancer, Gastroenterology, 127 (2004) 741-748.
[37] E. Saeland, A. I. Belo, S. Mongera, I. van Die, G. A. Meijer, Y. van Kooyk, Differential glycosylation of MUC1 and CEACAM5 between normal mucosa and tumour tissue of colon cancer patients, Int. J. Cancer, 131 (2012) 117-128.
[38] Y. Qiu, T. H. Patwa, L. Xu, K. Shedden, D. E. Misek, M. Tuck, G. Jin, M. T. Ruffin, D. K. Turgeon, S. Synal, Plasma glycoprotein profiling for colorectal cancer biomarker identification by lectin glycoarray and lectin blot, J. Proteome Res., 7 (2008) 1693-1703.
[39] C. Li, D. M. Simeone, D. E. Brenner, M. A. Anderson, K. A. Shedden, M. T. Ruffin, D. M. Lubman, Pancreatic cancer serum detection using a lectin/glyco-antibody array method, J. Proteome Res., 8 (2008) 483-492.
[40 ] J. Zhao, T. H. Patwa, W. Qiu, K. Shedden, R. Hinderer, D. E. Misek, M. A. Anderson, D. M. Simeone, D. M. Lubman, Glycoprotein microarrays with multi-lectin detection: unique lectin binding patterns as a tool for classifying normal, chronic pancreatitis and pancreatic cancer sera, J. Proteome Res., 6 (2007) 1864-1874.
[41] E. Miyoshi, M. Nakano, Fucosylated haptoglobin is a novel marker for pancreatic cancer: detailed analyses of oligosaccharide structures, Proteomics, 8 (2008) 3257-3262.
[42] S. Nie, A. Lo, J. Wu, J. Zhu, Z. Tan, D. M. Simeone, M. A. Anderson, K. A. Shedden, M. T. Ruffin, D. M. Lubman, Glycoprotein biomarker panel for pancreatic cancer discovered by quantitative proteomics analysis, J. Proteome Res., 13 (2014) 1873-1884.
[43] K. Kusama, Y. Okamoto, K. Saito, T. Kasahara, T. Murata, Y. Ueno, Y. Kobayashi, Y. Kamada, E. Miyoshi, Reevaluation of Pholiota squarrosa lectin-reactive haptoglobin as a pancreatic cancer biomarker using an improved ELISA system, Glycoconjugate J., (2017) 1-8.
[44] N. Parker, C. Makin, C. Ching, D. Eccleston, O. Taylor, D. Milton, J. M. Rhodes, A new enzyme-linked lectin/mucin antibody sandwich assay (CAM 17.1/WGA) assessed in combination with CA 19-9 and peanut lectin binding assay for the diagnosis of pancreatic cancer, Cancer, 70 (1992) 1062-1068.
[45] J. Y. Yiannakou, P. Newland, F. Calder, A. N. Kingsnorth, J. M. Rhodes, Prospective study of CAM 17 center dot 1/WGA mucin assay for serological diagnosis of pancreatic cancer, Lancet, 349 (1997) 389-392.
[46] T. Yue, I. J. Goldstein, M. A. Hollingsworth, K. Kaul, R. E. Brand, B. B. Haab, The prevalence and nature of glycan alterations on specific proteins in pancreatic cancer patients revealed using antibody-lectin sandwich arrays, Mol. Cel. Proteom., 8 (2009) 1697-1707.
[47] S. Pan, T. A. Brentnall, R. Chen, Glycoproteins and glycoproteomics in pancreatic cancer, World journal of gastroenterology, 22 (2016) 9288.
[48] K. Shimizu, K. Nakamura, S. Kobatake, S. Satomura, M. Maruyama, F. Kameko, J. Tajiri, R. Kato, The clinical utility of Lens culinaris agglutinin-reactive thyroglobulin ratio in serum for distinguishing benign from malignant conditions of the thyroid, Clin. Chim. Acta, 379 (2007) 101-104.
[49] T. Kanai, M. Amakawa, R. Kato, K. Shimizu, K. Nakamura, K. -i. Ito, Y. Hama, M. Fujimori, J. Amano, Evaluation of a new method for the diagnosis of alterations of Lens culinaris agglutinin binding of thyroglobulin molecules in thyroid carcinoma, Clinical chemistry and laboratory medicine, 47 (2009) 1285-1290.
[50] K. YAMAMOTO, T. TSUJI, O. TARUTANI, T. OSAWA, Structural changes of carbohydrate chains of human thyroglobulin accompanying malignant transformations of thyroid glands, The FEBS Journal, 143 (1984) 133-144.
[51] A. Naitoh, Y. Aoyagi, H. Asakura, Highly enhanced fucosylation of serum glycoproteins in patients with hepatocellular carcinoma, Journal of gastroenterology and hepatology, 14 (1999) 436-445.
[52] Y. H. Ahn, P. M. Shin, N. R. Oh, G. W. Park, H. Kim, J. S. Yoo, A lectin-coupled, targeted proteomic mass spectrometry (MRM MS) platform for identification of multiple liver cancer biomarkers in human plasma, J. Proteomics, 75 (2012) 5507-5515.
[53] Y.H. Ahn, P. M. Shin, Y. S. Kim, N. R. Oh, E. S. Ji, K. H. Kim, Y. J. Lee, S. H. Kim, J. S. Yoo, Quantitative analysis of aberrant protein glycosylation in liver cancer plasma by AAL-enrichment and MRM mass spectrometry, Analyst, 138 (2013) 6454-6462.
[54] J. H. Lee, C. H. Cho, S. H. Kim, J. G. Kang, J. S. Yoo, C. L. Chang, J. -H. Ko, Y. -S. Kim, Semi-quantitative measurement of a specific glycoform using a DNA-tagged antibody and lectin affinity chromatography for glyco-biomarker development, Mol. Cel. Proteom., 14 (2015) 782-795.
[55] H. Toyoda, T. Kumada, T. Tada, Y. Kaneoka, A. Maeda, F. Kanke, S. Satomura, Clinical utility of highly sensitive Lens culinaris agglutinin-reactive alpha-fetoprotein in hepatocellular carcinoma patients with alpha-fetoprotein<20 ng/ml, Cancer Sci., 102 (2011) 1025-1031.
[56] X. Yi, S. Yu, Y. Bao, Alpha-fetoprotein-L3 in hepatocellular carcinoma: a meta-analysis, Clin. Chim. Acta, 425 (2013) 212-220.
[57] M.A. Comunale, M. Wang, J. Hafner, J. Krakover, L. Rodemich, B. Kopenhaver, R. E. Long, O. Junaidi, A. M. D. Bisceglie, T. M. Block, Identification and development of fucosylated glycoproteins as biomarkers of primary hepatocellular carcinoma, J. Proteome Res., 8 (2008) 595-602.
[58] Y. Liu, J. He, C. Li, R. Benitez, S. Fu, J. Marrero, D. M. Lubman, Identification and Confirmation of Biomarkers Using an Integrated Platform for Quantitative Analysis of Glycoproteins and Their Glycosylations, J. Proteome Res., 9 (2010) 798-805.
[59] L. F. Hoagland, M. J. Campa, E. B. Gottlin, J. E. Herndon, E. F. Patz, Haptoglobin and posttranslational glycan-modified derivatives as serum biomarkers for the diagnosis of nonsmall cell lung cancer, Cancer, 110 (2007) 2260-2268.
[60] D. Wang, M. Hincapie, T. Rejtar, B. L. Karger, Ultrasensitive characterization of site-specific glycosylation of affinity-purified haptoglobin from lung cancer patient plasma using 10 μm id porous layer open tubular liquid chromatography-linear ion trap collision-induced dissociation/electron transfer dissociation mass spectrometry, Anal. Chem., 83 (2011) 2029-2037.
[61] H. Y. Tsai, K. Boonyapranai, S. Sriyam, C. J. Yu, S. W. Wu, K. H. Khoo, S. Phutrakul, S. T. Chen, Glycoproteomics analysis to identify a glycoform on haptoglobin associated with lung cancer, Proteomics, 11 (2011) 2162-2170.
[62] J. N. Arnold, R. Saldova, M. C. Galligan, T. B. Murphy, Y. Mimura-Kimura, J. E. Telford, A. K. Godwin, P. M. Rudd, Novel Glycan Biomarkers for the Detection of Lung Cancer, J. Proteome Res., 10 (2011) 1755-1764.
[63] Y. Hirao, H. Matsuzaki, J. Iwaki, A. Kuno, H. Kaji, T. Ohkura, A. Togayachi, M. Abe, M. Nomura, M. Noguchi, Glycoproteomics approach for identifying glycobiomarker candidate molecules for tissue type classification of non-small cell lung carcinoma, J. Proteome Res., 13 (2014) 4705-4716.
[64] M. Ferens-Sieczkowska, E. M. Kratz, B. Kossowska, E. Passowicz-Muszynska, R. Jankowska, Comparison of Haptoglobin and Alpha (1)-Acid Glycoprotein Glycosylation in the Sera of Small Cell and Non-Small Cell Lung Cancer Patients, Postep. Hig. Med. Dosw., 67 (2013) 828-836.
[65] Y. Q. Liang, T. R. Ma, A. Thakur, H. J. Yu, L. Gao, P. Y. Shi, X. T. Li, H. Ren, L. Y. Jia, S. Zhang, Z. Li, M. W. Chen, Differentially expressed glycosylated patterns of alpha-1-antitrypsin as serum biomarkers for the diagnosis of lung cancer, Glycobiology, 25 (2015) 331-340.
[66] S. D. Shiayan, V. V. Nasonov, N. V. Bovin, L. I. Novikova, V. A. Aleshkin, A. G. Lutov, STUDIES OF N-LINKED OLIGOSACCHARIDE CHAINS OF ALPHA (1)-ACID GLYCOPROTEIN ISOLATED FROM ASCITIC FLUID OF STOMACH-CANCER PATIENTS AND NORMAL SERUM, Eksperimentalnaya Onkologiya, 15 (1993) 53-61.
[67] J. Bones, J. C. Byrne, N. O'Donoghue, C. McManus, C. Scaife, H. Boissin, A. Nastase, P. M. Rudd, Glycomic and glycoproteomic analysis of serum from patients with stomach cancer reveals potential markers arising from host defense response mechanisms, J. Proteome Res., 10 (2010) 1246-1265.
[68] S. H. Lee, S. Jeong, J. Lee, I. S. Yeo, M. J. Oh, U. Kim, S. Kim, S. H. Kim, S. Y. Park, J. H. Kim, S. H. Park, J. H. Kim, H. J. An, Glycomic profiling of targeted serum haptoglobin for gastric cancer using nano LC/MS and LC/MS/MS, Mol. Biosyst., 12 (2016) 3611-3621.
[69] L. Valmu, H. Alfthan, K. Hotakainen, S. Birken, U. H. Stenman, Site-specific glycan analysis of human chorionic gonadotropin beta-subunit from malignancies and pregnancy by liquid chromatography-electrospray mass spectrometry, Glycobiology, 16 (2006) 1207-1218.
[70] T. Kamoto, S. Satomura, T. Yoshiki, Y. Okada, F. Henmi, H. Nishiyama, T. Kobayashi, A. Terai, T. Habuchi, O. Ogawa, Lectin-reactive alpha-fetoprotein (AFP-L3%) curability and prediction of clinical course after treatment of non-seminomatous germ cell tumors, Jpn. J. Clin. Oncol., 32 (2002) 472-476.
[71] S. Ambrose, N. Gordon, J. Goldsmith, W. Wei, M. Zeegers, N. James, M. Knowles, R. Bryan, D. Ward, Use of Aleuria alantia Lectin Affinity Chromatography to Enrich Candidate Biomarkers from the Urine of Patients with Bladder Cancer, Proteomes, 3 (2015) 266.
[72] R. Azevedo, A. Peixoto, C. Gaiteiro, E. Fernandes, M. Neves, L. Lima, L. L. Santos, J. A. Ferreira, Over forty years of bladder cancer glycobiology: Where do glycans stand facing precision oncology?, Oncotarget, 8 (2017) 91734-91764.
[73] E. Pocheć, A. Lityńska, M. Bubka, A. Amoresano, A. Casbarra, Characterization of the oligosaccharide component of α3β1 integrin from human bladder carcinoma cell line T24 and its role in adhesion and migration, European journal of cell biology, 85 (2006) 47-57.
[74] S. Cotton, R. Azevedo, C. Gaiteiro, D. Ferreira, L. Lima, A. Peixoto, E. Fernandes, M. Neves, D. Neves, T. Amaro, R. Cruz, A. Tavares, M. Rangel, A. M. N. Silva, L. L. Santos, J. A. Ferreira, Targeted O-glycoproteomics explored increased sialylation and identified MUC16 as a poor prognosis biomarker in advanced-stage bladder tumours, Mol. Oncol., 11 (2017) 895-912.
[75] N. Yang, S. Feng, K. Shedden, X. L. Xie, Y. S. Liu, C. J. Rosser, D. M. Lubman, S. Goodison, Urinary Glycoprotein Biomarker Discovery for Bladder Cancer Detection Using LC/MS-MS and Label-Free Quantification, Clin. Cancer Res., 17 (2011) 3349-3359.
[76] Yamashita K, Umetsu K, Suzuki T, Ohkura T (1992) Purification and characterization of a Neu5Ac alpha 2—>>6Gal beta 1—>4GlcNAc and HSO3(−)—>6Gal beta 1—>GlcNAc specific lectin in tuberous roots of Trichosanthes japonica. Biochemistry 31 (46):11647-11650.

The method of determining the glycoprofile of a protein of the invention provides information on the glycoprofile of said protein. This information is useful in the diagnosis of various diseases as described herein (c.f. also Table 1). Diseases, which are known to be characterized by proteins having an altered glycoprofile include, but are not limited to, cancer, autoimmune diseases, inflammatory diseases, or neurodegenerative diseases.

Accordingly, the present invention further relates to a method for diagnosing whether a subject may be at a risk or may suffer from cancer, comprising
(a) contacting a sample obtained from said subject comprising a cancer biomarker protein with first beads having coupled thereto an antibody directed against said cancer biomarker protein,
   to form an antibody-protein complex,
(b) contacting said antibody-protein complex with one or more further beads, each further bead having coupled thereto (i) a label and (ii) a lectin,
   to form an antibody-protein-lectin complex; and
(c) determining the glycoprofile of said cancer biomarker protein,
   wherein a deviation of said glycoprofile from the healthy glycoprofile of said cancer biomarker protein is indicative that said subject may be at a risk or may suffer from cancer.

Accordingly, the present invention further relates to a method for diagnosing whether a subject may be at a risk or may suffer from an autoimmune disease, comprising
(a) contacting a sample obtained from said subject comprising an autoimmune disease biomarker protein with first beads having coupled thereto an antibody directed against said autoimmune disease biomarker protein,
   to form an antibody-protein complex,
(b) contacting said antibody-protein complex with one or more further beads, each further bead having coupled thereto (i) a label and (ii) a lectin,
   to form an antibody-protein-lectin complex; and
(c) determining the glycoprofile of said autoimmune disease biomarker protein,
   wherein a deviation of said glycoprofile from the healthy glycoprofile of said autoimmune disease biomarker protein is indicative that said subject may be at a risk or may suffer from an autoimmune disease.

In the context of autoimmune diseases, a glycoprofile indicating that said subject may be at a risk or may suffer from an autoimmune disease may include increased desialysation, or (exposure of) GicNAc and mannose on antibodies, which may lead to to activation of the “alternative lectin pathway of complement activation” by MBP (mannose-binding protein.

Accordingly, the present invention further relates to a method for diagnosing whether a subject may be at a risk or may suffer from an inflammatory disease, comprising
(a) contacting a sample obtained from said subject comprising an inflammatory disease biomarker protein with first beads having coupled thereto an antibody directed against said inflammatory disease biomarker protein,
   to form an antibody-protein complex,
(b) contacting said antibody-protein complex with one or more further beads, each further bead having coupled thereto (i) a label and (ii) a lectin,
   to form an antibody-protein-lectin complex; and
(c) determining the glycoprofile of said inflammatory disease biomarker protein,
   wherein a deviation of said glycoprofile from the healthy glycoprofile of said inflammatory disease biomarker protein is indicative that said subject may be at a risk or may suffer from an inflammatory disease.

Accordingly, the present invention further relates to a method for diagnosing whether a subject may be at a risk or may suffer from a neurodegenerative disease, comprising
(a) contacting a sample obtained from said subject comprising a neurodegenerative disease biomarker protein with first beads having coupled thereto an antibody directed against said neurodegenerative disease biomarker protein,
   to form an antibody-protein complex,
(b) contacting said antibody-protein complex with one or more further beads, each further bead having coupled thereto (i) a label and (ii) a lectin,
   to form an antibody-protein-lectin complex; and
(c) determining the glycoprofile of said neurodegenerative disease biomarker protein,
   wherein a deviation of said glycoprofile from the healthy glycoprofile of said neurodegenerative disease biomarker protein is indicative that said subject may be at a risk or may suffer from a neurodegenerative disease.

A “healthy glycoprofile” of a biomarker protein describes the glycoprofile of said biomarker in a sample obtained from a healthy (human) subject, preferably not suffering from cancer, autoimmune disease, neurodegenerative disease, or inflammatory disease or any other particular disease described herein relating to cancer, autoimmune disease, inflammatory disease, or neurodegenerative disease.

The term “lectin” when used herein refers to a carbohydrate-binding protein. A lectin typically is highly specific for a carbohydrate moiety or carbohydrate moieties (e.g., it reacts specifically with terminal glycosidic residues of other molecules such as a glycan/s of a glycoprotein (e.g., branching sugar molecules of glycoproteins, e.g., such as target polypeptides within the meaning of the present invention and biomarkers as described in Table 1 herein). Lectins are commonly known in the art. A skilled person is readily available to determine which lectin may be used for binding a carbohydrate moiety or carbohydrate moieties of interest, e.g. a carbohydrate moiety or carbohydrate moieties of a glycan attached to a protein. Preferred lectins applied in the context of the present invention are described herein. Also included by the term “lectin” are Siglecs (sialic acid-binding immunoglobulin-like lectins), Galectins (lectins that bind specifically to β-galactoside containing glycans) and Selectins (bind to the sialyl Lewis X (SLe^{x}) determinant NeuAc═2-3Galβ1-4(Fucα1-3)GlcNAc and related sialylated, fucosylated glycans). Notably, the term “lectin” when used herein also refers to glycan-binding antibodies.

Accordingly, the term “lectin” when used herein may also encompass lectins, Siglecs, Galectins, Selectins, etc. as well as glycan-binding antibodies. Lectins may also include DNA/RNA aptamers recognizing glycans.

The lectin may be specific for core fucose, antennary fucose, Fucα1-6GlcNAc-N-Asn containing N-linked oligosaccharides, Fucα1-6/3GlcNAc, α-L-Fuc, Fucα1-2Galβ1-4(Fucα1-3)GlcNAc, Fucα1-2Gal, Fucα1-6GlcNAc, Manβ1-4GlcNAcβ1-4GlcNAc, branched N-linked hexa-saccharide, Manα1-3Man, α-D-Man, (GlcNAcβ1-4)₂₋₄, Galβ1-4GlcNAc, GlcNAcα1-4Galβ1-4GlcNAc, (GlcNAcβ1-4)₂₋₅, Neu5Ac (sialic acid), Galβ1-3GalNAc-serine/threonine, Galβ1-3GalNAc, Galβ1-6Gal, Galβ1-4GlcNAc, Galβ1-3GalNAc, GalNAcα1-3GalNAc, GalNAcα1-3Gal, GalNAcα/β1-3/4Gal, α-GalNAc, GalNAcβ1-4Gal, GalNAcα1-3(Fucα1-2)Gal, GalNAcα1-2Gal, GalNAcα1-3GalNAc, GalNAcβ1-3/4Gal, GalNAc-Ser/Thr (Tn antigen), Galβ1-3GalNAc-Ser/Thr (T antigen), GalNAcβ1-4GlcNAc (LacdiNAc), α-2,3Neu5Ac (α2-3 linked sialic acid), α-2,6Neu5Ac (α2-6 linked sialic acid), α-2,8Neu5Ac (α2-8 linked sialic acid), sialic acid (α-2,3Neu5Ac, α-2,6Neu5Ac or α-2,8Neu5Ac), Neu5Acα4/9-O-Ac-Neu5Ac, Neu5Acα2-3Galβ1-4Glc/GlcNAc, Neu5Acα2-6Gal/GalNAc, N-linked bi-antennary, N-linked tri/tetra-antennary, branched β1-6GlcNAc, Galα1-3(Fucα1-2)Galβ1-3/4GlcNAc, Galβ1-3(Fucα1-4)GlcNAc, NeuAcα2-3Galβ1-3(Fucα1-4)GlcNAc, Fucα1-2Galβ1-3(Fucα1-4)GlcNAc, Galβ1-4(Fucα1-3)GlcNAc, NeuAcα2-3Galβ1-4(Fucα1-3)GlcNAc, Fucα1-2Galβ1-4(Fucα1-3)GlcNAc, high mannose, sialyl Lewis^{a} (sialyl Le^{a}) antigen, sialyl Lewis^{x} (sialyl Le^{x}) antigen, Lewis^{x} (Le^{x}) antigen, sialyl Tn antigen, sialyl T antigen, Lewis^{y} (Le^{y}) antigen, sulfated core1 glycan, Tn antigen, T antigen, core 2 glycan, Lewis^{a} (Le^{a}) antigen, (GlcNAcβ1-4)ₙ, β-D-GlcNAc, GalNAc, Gal-GlcNAc, GlcNAc, Galα1-3Gal, Galβ1-3GalNAc, α-Gal, α-GalNAc, (GlcNAc)ₙ, or branched (LacNAc)ₙ.

Carbohydrate abbreviations as used herein include: “Neu5Ac” for N-acetylneuraminic acid; “Fuc” for fucose, “GalNAc” for N-acetylgalactosamine; “GlcNAc” for N-acetylglucosamine; “Gal” for galactose (e.g., Varki A, Cummings R D, Esko J D, Freeze H H, Stanley P, Bertozzi C R, Hart G W, E. M E., Essentials of Glycobiology, 2nd edition, Cold Spring Harbor Laboratory Press, Cold Spring Harbor (NY), 2009).

Furthermore, as used herein the following terms are defined below:
“core fucose” means fucose is linked via an α-glycosidic bond of its C1 atom to the C6 atom of N-acetylglucosamine,
“antennary fucose” means fucose is linked via an α-glycosidic bond of its C1 atom to the C3 atom of N-acetylglucosamine or fucose is linked via an α-glycosidic bond of its C1 atom to the C2 atom of neighboring fucose,
“Fucα1-6GlcNAc-N-Asn containing N-linked oligosaccharides” means oligosaccharides which have fucose linked via a α-glycosidic bond of its C1 atom to the C6 atom of N-acetylglucosamine, which is linked to asparagine via N-glycosidic bond,
“Fucα1-6/3GlcNAc” means fucose is linked via a α-glycosidic bond of its C1 atom to the C6 (C3) atom of N-acetylglucosamine,
“α-L-Fuc” means α-L-fucose,
“Fucα1-2Galβ1-4(Fucα1-3)GlcNAc” means fucose is linked via an α-glycosidic bond of its C1 atom to the C2 atom of galactose, which is linked via an β0 glycosidic bond of its C1 atom to the C4 atom of N-acetylglucosamine; at the same time second fucose is linked via an α-glycosidic bond of its C1 atom to the C3 atom of N-acetylglucosamine,
“Fucα1-2Gal” means fucose is linked via a α-glycosidic bond of its C1 atom to the C2 atom of galactose,
“Fucα1-6GlcNAc” means fucose is linked via a α-glycosidic bond of its C1 atom to the C6 atom of N-acetylglucosamine,
“Manβ1-4GlcNAcβ1-4GlcNAc” means mannose is linked via a β-glycosidic bond of its C1 atom to the C4 atom of N-acetylglucosamine, which is linked via a β-glycosidic bond of its C1 atom to the C4 atom of N-acetylglucosamine,
“branched N-linked hexa-saccharide” means non-linear glycan composed of several carbohydrates linked to asparagine by N-glycosidic bond
“Manα1-3Man” means mannose is linked via a α-glycosidic bond of its C1 atom to the C3 atom of mannose,
“α-D-Man” means α-D-mannose,
“(GlcNAcβ1-4)₂₋₄” means N-acetylglucosamine is linked via a β-glycosidic bond of its C1 atom to the C4 atom of N-acetylglucosamine repeatedly,
“Galβ1-4GlcNAc” means galactose is linked via a β-glycosidic bond of its C1 atom to the C4 atom of N-acetylglucosamine,
“GlcNAcα1-4Galβ1-4GlcNAc” means N-acetylglucosamine is linked via a α-glycosidic bond of its C1 atom to the C4 atom of galactose, which is linked via a β-glycosidic bond of its C1 atom to the C4 atom of N-acetylglucosamine,
“N-acetylglucosamine” means amide between glucosamine and acetic acid,
“(GlcNAcβ1-4)₂₋₅” means N-acetylglucosamine is linked via a β-glycosidic bond of its C1 atom to the C4 atom of N-acetylglucosamine repeatedly,
“Neu5Ac” (or sialic acid) means N-acetylneuraminic acid,
“Galβ1-3GalNAc-serine/threonine” means galactose is linked via a β-glycosidic bond of its C1 atom to the C3 atom of N-acetylglucosamine, which is linked to serine/threonine,
“Galα1-3GalNAc” means galactose is linked via a α-glycosidic bond of its C1 atom to the C3 atom of N-acetylgalactosamine,
“Galβ1-6Gal” means galactose is linked via a β-glycosidic bond of its C1 atom to the C6 atom of galactose,
“Galβ1-4GlcNAc” means galactose is linked via a β-glycosidic bond of its C1 atom to the C3 atom of N-acetylglucosamine,
“Galβ1-3GalNAc” means galactose is linked via a β-glycosidic bond of its C1 atom to the C3 atom of N-acetylgalactosamine,
“GalNAcα1-3GalNAc” means N-acetylgalactosamine is linked via a α-glycosidic bond of its C1 atom to the C3 atom of N-acetylgalactosamine,
“GalNAcα1-3Gal” means N-acetylgalactosamine is linked via a α-glycosidic bond of its C1 atom to the C3 atom of galactose,
“GalNAcα/β1-3/4Gal” means N-acetylgalactosamine is linked via a α- or β-glycosidic bond of its C1 atom to the C3 or C4 atom of galactose,
“α-GalNAc” means amide between α-galactosamine and acetic acid,
“GalNAcβ1-4Gal” means N-acetylgalactosamine is linked via a β-glycosidic bond of its C1 atom to the C4 atom of galactose,
“GalNAcα1-3(Fucα1-2)Gal” means N-acetylgalactosamine is linked via a α-glycosidic bond of its C1 atom to the C3 atom of galactose, at the same time fucose is linked via a α-glycosidic bond of its C1 atom to the C2 atom of galactose,
“GalNAcα1-2Gal” means N-acetylgalactosamine is linked via a α-glycosidic bond of its C1 atom to the C3 atom of galactose,
“GalNAcα1-3GalNAc” means N-acetylgalactosamine is linked via a α-glycosidic bond of its C1 atom to the C3 atom of N-acetylgalactosamine,
“GalNAcβ1-3/4Gal” means N-acetylgalactosamine is linked via a β-glycosidic bond of its C1 atom to the C3 or C4 atom of galactose,
“GalNAc-Ser/Thr” (or Tn antigen,) means N-acetylgalactosamine is linked to serine/threonine via O-glycosidic bond,
“Galβ1-3GalNAc-Ser/Thr” (T antigen or Thomsen-Friedenreich antigen) means galactose is linked via a β-glycosidic bond of its C1 atom to the C3 atom of N-acetylgalactosamine, which is linked to serine/threonine via O-glycosidic bond,
“GalNAcβ1-4GlcNAc” (or LacdiNAc) means N-acetylgalactosamine is linked via a β-glycosidic bond of its C1 atom to the C4 atom of N-acetylglucosamine,
“α2-3Neu5Ac” (or α2-3-linked sialic acid) means N-acetylneuraminic acid is linked via a α-glycosidic bond of its C2 atom to the C3 atom of a neighboring saccharide,
“α2-6Neu5Ac” (or α2-6-linked sialic acid) means N-acetylneuraminic acid is linked via a α-glycosidic bond of its C2 atom to the C6 atom of a neighboring saccharide,
“α2-8Neu5Ac” (or α2-8-linked sialic acid) means N-acetylneuraminic acid is linked via a α-glycosidic bond of its C2 atom to the C8 atom of a neighboring N-acetylneuraminic acid,
“Neu5Acα4/9-O-Ac-Neu5Ac” means N-acetylneuraminic acid is linked via a α-glycosidic bond of its C4 atom to the C9 atom of a neighboring O-acetyl N-acetylneuraminic acid,
“Neu5Acα2-3Galβ1-4Glc/GlcNAc” means N-acetylneuraminic acid is linked via a α-glycosidic bond of its C2 atom to the C3 atom of galactose, which is linked via β-glycosidic bond of its C1 atom to the C4 atom of glucose or N-acetylglucosamine,
“Neu5Acα2-6Gal/GalNAc” means N-acetylneuraminic acid is linked via a α-glycosidic bond of its C2 atom to the C6 atom of galactose or N-acetylgalactosamine,
“N-linked bi-antennary” means non-linear glycan with two antennas (carbohydrate chains) linked to asparagine by N-glycosidic bond,
“N-linked tri/tetra-antennary” means non-linear glycan with three/tetra antennas (carbohydrate chains) linked to asparagine by N-glycosidic bond,
“branched β1-6GlcNAc” means N-acetylglucosamine is linked via a β-glycosidic bond of its C1 atom to the C6 atom of neighboring saccharide,
“Galα1-3(Fucα1-2)Galβ1-3/4GlcNAc” means galactose is linked via a α-glycosidic bond of its C1 atom to the C3 atom of galactose, which is linked via a β-glycosidic bond of its C1 atom to the C3 or C4 atom of N-acetylglucosamine; at the same time fucose is linked via a α-glycosidic bond of its C1 atom to the C2 atom of N-acetylglucosamine,
“Galβ1-3(Fucα1-4)GlcNAc” means galactose is linked via a β-glycosidic bond of its C1 atom to the C3 atom of N-acetylglucosamine; at the same time fucose is linked via α-glycosidic bond of its C1 atom to the C4 atom of N-acetylglucosamine,
“NeuAcα2-3Galβ1-3(Fucα1-4)GlcNAc” means N-acetylneuraminic acid is linked via a α-glycosidic bond of its C2 atom to the C3 atom of galactose, which is linked via β-glycosidic bond of its C1 atom to the C3 atom N-acetylglucosamine; at the same time fucose is linked via a α-glycosidic bond of its C1 atom to the C4 atom of N-acetylglucosamine,
“Fucα1-2Galβ1-3(Fucα1-4)GlcNAc” means fucose is linked via a α-glycosidic bond of its C1 atom to the C2 atom of galactose, which is linked via β-glycosidic bond of its C1 atom to the C3 atom N-acetylglucosamine; at the same time second fucose is linked via α-glycosidic bond of its C1 atom to the C4 atom of N-acetylglucosamine,
“Galβ1-4(Fucα1-3)GlcNAc” means galactose is linked via β-glycosidic bond of its C1 atom to the C4 atom of N-acetylglucosamine; at the same time fucose is linked via α-glycosidic bond of its C1 atom to the C3 atom of N-acetylglucosamine,
“NeuAcα2-3Galβ1-4(Fucα1-3)GlcNAc” means N-acetylneuraminic acid is linked via α-glycosidic bond of its C2 atom to the C3 atom of galactose, which is linked via β-glycosidic bond of its C1 atom to the C4 atom N-acetylglucosamine; at the same time fucose is linked via α-glycosidic bond of its C1 atom to the C3 atom of N-acetylglucosamine,
“Fucα1-2Galβ1-4(Fucα1-3)GlcNAc” means fucose is linked via α-glycosidic bond of its C1 atom to the C2 atom of galactose, which is linked via β-glycosidic bond of its C1 atom to the C4 atom N-acetylglucosamine; at the same time second fucose is linked via α-glycosidic bond of its C1 atom to the C3 atom of N-acetylglucosamine,
“high mannose” means glycan containing more than three mannose units,
“sialyl Lewis^{a}” (sialyl Le^{a}) antigen is Neu5Acα2-3/6Galβ1-3(Fucα1-4)GlcNAc meaning N-acetylneuraminic acid is linked via α-glycosidic bond of its C2 atom to the C3 or C6 atom of galactose, which is linked via β-glycosidic bond of its C1 atom to the C3 atom N-acetylglucosamine; at the same time fucose is linked via α-glycosidic bond of its C1 atom to the C4 atom of N-acetylglucosamine,
“sialyl Lewis^{x}” (sialyl Le^{x}) antigen is Neu5Acα2-3/6Galβ1-4(Fucα1-3)GlcNAc meaning N-acetylneuraminic acid is linked via α-glycosidic bond of its C2 atom to the C3 or C6 atom of galactose, which is linked via β-glycosidic bond of its C1 atom to the C4 atom N-acetylglucosamine; at the same time fucose is linked via α-glycosidic bond of its C1 atom to the C3 atom of N-acetylglucosamine,
“Lewis^{x}” (Le^{x}) antigen is “Galβ1-4(Fucα1-3)GlcNAc” meaning galactose is linked via β-glycosidic bond of its C1 atom to the C4 atom of N-acetylglucosamine; at the same time fucose is linked via α-glycosidic bond of its C1 atom to the C3 atom of N-acetylglucosamine,
“sialyl Tn antigen” is “Neu5Acα2-3/6GalNAc-Ser/Thr” meaning N-acetylneuraminic acid is linked via α-glycosidic bond of its C2 atom to the C3 or C6 atom of N-acetylgalactosamine, which is linked to serine/threonine via O-glycosidic bond,
“sialyl T antigen” is “Neu5Acα2-3/6Galβ1-3GalNAc-Ser/Thr” meaning N-acetylneuraminic acid is linked via α-glycosidic bond of its C2 atom to the C3 or C6 atom of galactose, which is linked via β-glycosidic bond of its C1 atom to the C3 atom of N-acetylgalactosamine, which is linked to serine/threonine via O-glycosidic bond,
“Lewis^{y}” (Le^{y}) antigen is “Fucα1-2Galβ1-4(Fucα1-3)GlcNAc” meaning fucose is linked via α-glycosidic bond of its C1 atom to the C2 atom of galactose, which is linked via β-glycosidic bond of its C1 atom to the C4 atom of N-acetylglucosamine; at the same time second fucose is linked via α-glycosidic bond of its C1 atom to the C3 atom of N-acetylglucosamine,
“sulfated core1 glycan” is a glycan based on sulfated extended form of T antigen,
“core 2 glycan” is a glycan based on an extended form of Galβ1-3(GlcNAcβ1-6)GalNAc-Ser/Thr meaning an extended form of glycan having galactose linked via β-glycosidic bond of its C1 atom to the C3 atom of N-acetylgalactosamine, at the same time N-acetylglucosamine is linked via β-glycosidic bond of its C1 atom to the C6 atom of N-acetylgalactosamine, which is linked to serine/threonine
“Lewis^{a}” (Le^{a}) antigen is Galβ1-3(Fucα1-4)GlcNAc meaning galactose is linked via β-glycosidic bond of its C1 atom to the C3 atom N-acetylglucosamine; at the same time fucose is linked via α-glycosidic bond of its C1 atom to the C4 atom of N-acetylglucosamine,
“(GlcNAcβ1-4)ₙ” means N-acetylglucosamine is linked via β-glycosidic bond of its C1 atom to the C4 atom of N-acetylglucosamine repeatedly,
“β-D-GlcNAc” means amide between β-D-glucosamine and acetic acid,
“GalNAc” means amide between galactosamine and acetic acid i.e. N-acetylgalactosamine,
“Gal-GlcNAc” means galactose is linked to N-acetylglucosamine via non-specified linkage,
“GlcNAc” means amide between glucosamine and acetic acid i.e. N-acetylglucosamine.
“Galα1-3Gal” means galactose is linked via α-glycosidic bond of its C1 atom to the C3 atom of galactose,
“Galβ1-3GalNAc” means galactose is linked via β-glycosidic bond of its C1 atom to the C3 atom of N-acetylgalactosamine,
“α-Gal” means α-galactose,
“α-GalNAc” means amide between α-D-galactosamine and acetic acid,
“(GlcNAc)ₙ” means N-acetylglucosamine is linked to N-acetylglucosamine via non-specified linkage,
“branched (LacNAc)ₙ” is branched and repeated form of Galβ1,4-GlcNAc meaning a branched and repeated form of galactose linked via β-glycosidic bond of its C1 atom to the C4 atom of N-acetylglucosamine.

Lectins can be obtained from seeds of leguminous plants, but also from other plant and animal sources. Lectins can contain binding sites for specific mono- and oligosaccharides (e.g., glycans of glycoproteins). They can agglutinate cells by binding to specific sugar residues in membrane glycoproteins. Preferably, lectins of the present invention are selected from the group consisting of: Maackia amurensis lectin II (MAA II); Concanavalin A (Con A); Aleuria aurantia lectin (AAL); Sambucus nigra (SNA-I) lectin; Wisteria floribunda lectin (WFL) as defined herein.

Further preferred lectins of the present invention are shown in Table 1 below. In this context, the lectins do not necessarily have to be used in combination with the antibodies or proteins shown but also can be seen as examples of pairs of lectins and their recognized glycan structure.

Particularly preferred lectins of the present invention are lectins with the following UniProtKB Accession Numbers (sequence according to v1 of the sequence in the database): P0DKL3, P02866, P18891, O04366, A0A218PFP3, Q945S3, Q00022, Q6YNX3, Q71QF2, P02872, P18670, Q2UNX8, Q8L5H4, A0A089ZWN7, P05045, P19588, P83410, P17931, P56470, P24146, Q41263, Q39990, Q2F1K8, G9M5T0, B3XYC5, P02870, P19664, PODKL3, P49300, A9XX86, Q40423, P16300, P05088, P05087, Q9AVB0, P02867, O24313, Q9SM56, P06750, B9SPG3, Q9BZZ2, P20916, Q9NYZ4, Q96RL6, P05046, P93535, P02876, P10968, P10969, P22972 or P56625 as well as corresponding mature forms thereof.

Exemplary lectins of the present invention further include:
Maackia amurensis lectin II (MAA II) is the hemagglutinin isolectin from Maackia seeds. Sialic acid-binding lectin recognizing oligosaccharides containing terminal sialic acid linked via α2-3 bond to neighbouring galactose residues. Binds the trisaccharide sequence Neu5Acα2-3-Gal-β-1-4-GlcNAc. Preferably, MAA II has a SEQ ID NO: 2 (or its mature form).
Concanavalin A (Con A) a D-mannose specific lectin originally extracted from the jack-bean, Canavalia ensiformis. Preferably, Con A has a SEQ ID NO: 3 or SEQ ID NO: 4 (Con A, mature form).
Aleuria aurantia lectin (AAL) is a fucose-specific lectin extracted from Aleuria aurantia (Orange peel mushroom). Preferably, AAL has a SEQ ID NO: 5 (or its mature form). The isolation of AAL is, for example, described in (Debray et al., Kochibe et al.).
Sambucus nigra (SNA-I) lectin is a Neu5Acα2-6)Gal/GalNAc specific agglutinin extracted from Sambucus nigra (European elder). Preferably, SNA-I has a SEQ ID NO: 6 (or its mature form).
Wisteria floribunda lectin (WFL) is an agglutinin extracted from Wisteria floribunda (Japanese wisteria). Preferably, WFL has a SEQ ID NO: 7 (or its mature form).

Furthermore, suitable lectins within the meaning of the present invention may explicitly include post-translationally processed- and mature forms of the lectins as disclosed herein.

As used herein, the term “bead” refers to a small spherical object, e.g., made of glass, plastic, metal, agarose, latex, metallic nano- or microparticle, metal oxide nano- or microparticle or magnetic material. Accordingly, said first and/or further beads preferably is/are made of glass, plastic, metal, agarose, latex, metallic nano- or microparticle, metal oxide nano- or microparticle or magnetic material. Preferably, the first bead is a magnetic carrier. Preferably, the further bead is a magnetic carrier. As used herein, the term “magnetic carrier” refers to particles or beads comprising magnetic material or substance (e.g., iron or ferritin). Preferably, the magnetic carrier is a magnetic particle or magnetic bead (e.g., a ferritin conjugate). However, for avoidance of doubt, the magnetic carrier when referred herein is not a solid surface, such as a plate, e.g. a ELISA plate or microtiter plate.

As described herein, the one or more further beads comprise a label. Said label preferably is a detectable label. Preferred labels include, but are not limited to, an enzyme, a radioisotope, a fluorescent protein, a fluorescent dye, a bioluminescent label or a tag (e.g., biotin). The detectable labels can be any of the various types used currently in the field of in vitro diagnostics, including particulate labels including metals such as colloidal gold, isotopes, chromophores including fluorescent markers, biotin, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, polyHRP, alkaline phosphatase and the like, preferably horseradish peroxidase. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-star® or other luminescent substrates well-known to those in the art, for example the chelates of suitable lanthanides such as Terbium (III) and Europium (III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice. Accordingly, the label of said one or more further beads may be detected based on optical, fluorescent, luminescent, electrochemiluminescent and/or multi-analyte profiling (xMAP) readouts or means. The label of said one or more further beads may be detected by optical means such as absorption at a particular wavelength or inspection by the naked eye. The label of said one or more further beads may be detected by fluorescent means such as determining the emission of a fluorophore at a specific wavelength after excitation at a different, typically shorter, wave length. The label of said one or more further beads may be detected by electro chemiluminescent means, e.g., making of use the commercially available ELECSYS system by Roche. The label of said one or more further beads may be detected by multi-analyte profiling (xMAP), e.g., as described in WO 2007/075891.A “tag” as used herein may include, but is not limited to, affinity tags that are appended to proteins so that they can be purified from their crude biological source using an affinity technique such as chitin binding protein (CBP), maltose binding protein (MBP), Strep-tag and glutathione-S-transferase (GST) or the poly (His) tag is a widely used protein tag, which binds to metal matrices; chromatography tags that are used to alter chromatographic properties of the protein to afford different resolution across a particular separation technique such as FLAG-tag; epitope tags that are short peptide sequences which are chosen because high-affinity antibodies can be reliably produced in many different species such as ALFA-tag, V5-tag, Myc-tag, HA-tag, Spot-tag, T7-tag and NE-tag; fluorescence tags that are used to give visual readout on a protein such as GFP and its variants; protein tags that may allow specific enzymatic modification (such as biotinylation by biotin ligase) or chemical modification (such as reaction with FIASH-EDT2 for fluorescence imaging).

Preferably, the label is a microperoxidase. As used herein, the term “microperoxidase” or “MP” refers to a heme containing peptide portion of cytochrome c (e.g., shown as SEQ ID NO: 11, cytochrome c derived from Equus caballus, NCBI Reference Sequence: NP_001157486.1) that retains peroxidase activity (e.g., EC 1.11.1.7 enzymatic activity, e.g., microperoxidase-11).

Preferably, the heme containing peptide portion of cytochrome c is at least 60% or more (e.g., at least 65%, at least 70%, at least 75%, at least 80%, at least 85%, at least 90%, at least 95%, at least 96%, at least 97%, at least 98%, at least 99% or 100%) identical to a polypeptide sequence selected from the group consisting of: SEQ ID NO: 8 (MP-11 peptide), SEQ ID NO: 9 (MP-9 peptide) and SEQ ID NO: 10 (MP-8 peptide), preferably said microperoxidase (MP) peptide is selected from the group consisting of: SEQ ID NO: 8 (MP-11 peptide), SEQ ID NO: 9 (MP-9 peptide) and SEQ ID NO: 10 (MP-8 peptide).

A further suitable tag is biotin. Accordingly, the label preferably is biotin. In this embodiment, biotin can act as anchor for the addition of a further label, which can bind to biotin. Such a further label can in principle be any label described herein-obviously not biotin itself. Thus, instead of coupling the label directly to the one or more further beads, the (further) label can be “indirectly” coupled to the one or more further beads by binding to biotin. Thereby, the amplification effect described herein may also be achieved. Preferably, said further label that is or can be bound to biotin on the one or more further beads comprises a biotin-binding moiety such as streptavidin. Also envisioned is that the tag on the one or more further beads is a binding partner A such as biotin, wherein the further label comprises a ligand B capable of specifically binding to the binding partner A such as streptavidin. “Streptavidin” is a protein purified from the bacterium Streptomyces avidinii. Streptavidin homo-tetramers have an extraordinarily high affinity for biotin (also known as vitamin B7 or vitamin H). With a dissociation constant (K_{d}) on the order of around 10⁻¹⁴ mol/L, the binding of biotin to streptavidin is one of the strongest non-covalent interactions known in nature. An exemplary amino acid sequence of a wild type streptavidin is: MRKIVVAAIAVSLTTVSITASASADPSKDSKAQVSAAEAGITGTWYNQLGSTFIVTAGADGALTG TYESAVGNAESRYVLTGRYDSAPATDGSGTALGWTVAWKNNYRNAHSATTWSGQYVGGAEA RINTQWLLTSGTTEANAWKSTLVGHDTFTKVKPSAASIDAAKKAGVNNGNPLDAVQQ (SEQ ID NO: 1). An exemplary wild type sequence of streptavidin is also shown in UniProt database entry P22629, version 1 of 1 Aug. 1991. Streptavidin as used herein, e.g., in the context of the methods or uses described herein, may also encompass streptavidin muteins. Streptavidin muteins are, e.g., disclosed in WO 2017/186669 or WO 2014/076277. Streptavidin or streptavidin muteins used in the methods and uses of the invention may be derived from streptavidin variants which are shortened at the N- or/and the C-terminus. A preferred polypeptide according to the present invention comprises the amino acid sequence of a minimal streptavidin which begins N-terminally in the region of the amino acid positions 10 to 16 and terminates C-terminally in the region of the amino acid positions 133 to 142. Such a streptavidin mutein polypeptide corresponds preferably to a minimal streptavidin outside of the mutation region which comprises an amino acid sequence from position Ala13 to Ser139 and optionally has an N-terminal methionine residue instead of Ala13. In this application the numbering of amino acid positions refers throughout to the numbering of mature wt-streptavidin (Argarana et al., Nucleic Acids Res. 14 (1986), 1871-1882, cf. SEQ ID NO: 1) which is also deposited under accession number UniProtKB-P22629, v1 of 1 Aug. 1991. “Streptavidin” as used here, in the context of the methods or uses described herein, may also relate to other biotin-binding moieties besides streptavidin, e.g. proteins or aptamers binding to biotin.

In an embodiment, wherein the label is biotin and the further label is bound to biotin instead of directly to the further beads, it is possible that the actual detectable label is added to the antibody-protein-lectin complex of step (b) after step (b), e.g., in the optional step (b′) or in step (c).

An “antibody” when used herein is a protein comprising one or more polypeptides (comprising one or more binding domains, preferably antigen binding domains) substantially or partially encoded by immunoglobulin genes or fragments of immunoglobulin genes. Preferably, an antibody which is directed against a protein whose glycoprofile is determined as described herein, is not directed against a glycan attached to said protein. Put differently, an antibody which is directed against a protein whose glycoprofile is determined as described herein is preferably directed against the protein as such, i.e., is directed against an epitope within the amino acid sequence of said protein. The epitope may be a linear or conformational epitope. It may be a continuous or discontinuous epitope. The term “immunoglobulin” (Ig) is used interchangeably with “antibody” herein. The recognized immunoglobulin genes include the kappa, lambda, alpha, gamma, delta, epsilon and mu constant region genes, as well as myriad immunoglobulin variable region genes. In particular, an “antibody” when used herein, is typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, lgG3, lgG4, IgA1, and IgA2, with IgG being preferred in the context of the present invention. An antibody of the present invention is also envisaged which has an IgE constant domain or portion thereof that is bound by the Fc epsilon receptor I. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each light chain includes an N-terminal variable (V) domain (VL) and a constant (C) domain (CL). Each heavy chain includes an N-terminal V domain (VH), three or four C domains (CHs), and a hinge region. The constant domains are not involved directly in binding an antibody to an antigen, but can exhibit various effector functions, such as participation of the antibody dependent cellular cytotoxicity (ADCC). If an antibody should exert ADCC, it is preferably of the IgG1 subtype, while the IgG4 subtype would not have the capability to exert ADCC.

The term “antibody” also includes, but is not limited to, monoclonal, monospecific, poly- or multi-specific antibodies such as bispecific antibodies, humanized, camelized, human, single-chain, chimeric, synthetic, recombinant, hybrid, mutated, grafted, and in vitro generated antibodies, with chimeric or humanized antibodies being preferred. The term “humanized antibody” is commonly defined for an antibody in which the specificity encoding CDRs of HC and LC have been transferred to an appropriate human variable frameworks (“CDR grafting”). The term “antibody” also includes scFvs, single chain antibodies, diabodies or tetrabodies, domain antibodies (dAbs) and nanobodies. In terms of the present invention, the term “antibody” shall also comprise bi-, tri- or multimeric or bi-, tri- or multifunctional antibodies having several antigen binding sites. Said term also includes antigen binding portion(s). Also included by the term “antibody” may be FN3 scaffold, adnectin, affibody, anticalin, avimer, a bicyclic peptide, DARPin, a Kunitz domain, an Obody or an aptamer, such as a DNA, RNA or peptide aptamer.

Preferred antibodies of the present invention include, but are not limited to, an anti-PSA, anti-AFP, anti-MUC16, anti-WFDC2, anti-MUC1, anti-ERBB2, anti-CEACAM5, anti-FUT3 or anti-TG antibodies etc. Further preferred antibodies relating to the present invention are shown in Fehler! Verweisquelle konnte nicht gefunden werden. below.

Furthermore, the term “antibody” as employed in the invention also relates to derivatives of the antibodies (including fragments) described herein. A “derivative” of an antibody comprises an amino acid sequence which has been altered by the introduction of amino acid residue substitutions, deletions or additions. Additionally, a derivative encompasses antibodies which have been modified by a covalent attachment of a molecule of any type to the antibody or protein. Examples of such molecules include sugars, PEG, hydroxyl-, ethoxy-, carboxy- or amine-groups but are not limited to these. In effect the covalent modifications of the antibodies lead to the glycosylation, pegylation, acetylation, phosphorylation, amidation, without being limited to these.

As used herein, the term “specifically binds” or “directed against” refers to antibodies or fragments or derivatives thereof that specifically bind to a target glycoprotein or target polypeptide and do not specifically bind to another protein or polypeptide. The antibodies or fragments or derivatives thereof according to the invention bind to their respective targets through the variable domain of the antibody. Typically, binding is considered specific when the binding affinity is higher than 10⁻⁶ M. Preferably, binding is considered specific when binding affinity is about 10⁻¹¹ to 10⁻⁸ M (KD), preferably of about 10⁻¹¹ to 10⁻⁹ M. If necessary, nonspecific binding can be reduced without substantially affecting specific binding by varying the binding conditions. In case of binding of glycans to lectins the binding affinity is preferably in the range 10⁻³ to 10⁻⁶ (K_{D}). The methods of measuring corresponding K_{D}s for binding of glycans to lectins are known in the art and are readily available to a person skilled in the art.

As outlined herein, an antibody is bound to the first beads and a lectin and a label is coupled to the one or more further beads. The chemistry of coupling antibodies, lectins and labels to beads is well known to the person skilled in the art. Antibodies can, e.g., be coupled to beads coated with Protein A, Protein G or Protein L, coated with secondary antibodies or epoxy-coated beads. Antibodies, labels and lectins can, e.g., be coupled to beads coated with streptavidin or fusion tags. Alternatively or additionally, antibodies can be covalently coupled to beads modified by terminal-COOH groups using amine coupling chemistry (e.g., using 1-ethyl-3-(3-dimethylaminopropyl) carbodiimide (EDC) or N-hydroxysuccinimide (NHS) coupling chemistry). Such beads are commercially available, e.g., Dynabeads® from Thermo Fisher Scientific, Waltham, Massachusetts, USA.

The present invention further relates to a kit for performing the method of for diagnosing whether a subject may be at a risk or may suffer from cancer of the invention, comprising an antibody specific for a cancer biomarker protein as defined herein and one or more lectins as defined herein.

The present invention further relates to a kit for performing the method for diagnosing whether a subject may be at a risk or may suffer from an autoimmune disease of the invention, comprising an antibody specific for an autoimmune disease biomarker protein which is IgG and one or more lectins as defined herein.

The present invention further relates to a kit for performing the method for diagnosing whether a subject may be at a risk or may suffer from an inflammatory disease of the invention, comprising an antibody specific for an inflammatory biomarker protein which is IgG, IgA or CRP and one or more lectins as defined herein.

The present invention further relates to a kit for performing the method for diagnosing whether a subject may be at a risk or may suffer from a neurodegenerative disease of the invention, comprising an antibody specific for an inflammatory biomarker protein, preferably α-synuclein, tau-protein or amyloid beta protein and its isoforms, and one or more lectins as defined herein.

In a further embodiment of the invention, there are provided articles of manufacture and kits containing antibody or antigen binding portion thereof which can be used, for instance, for the therapeutic or non-therapeutic applications described above. The article of manufacture comprises a container with a label. Suitable containers include, for example, bottles, vials, and test tubes. The containers may be formed from a variety of materials such as glass or plastic. The container holds a composition which includes an active agent that is effective for therapeutic or non-therapeutic applications, such as described above. The active agent in the composition is the antibody or antigen binding portion thereof. The label on the container indicates that the composition is used for a specific therapy or non-therapeutic application and may also indicate directions for either in vivo or in vitro use, such as those described above.

The kits of the invention will typically comprise the container described above and one or more other containers comprising materials desirable from a commercial and user standpoint, including buffers, diluents, filters, needles, syringes, and package inserts with instructions for use.

The present invention is also characterized by the following items:
1. A method of determining the glycoprofile of a protein, comprising
(a) contacting a sample comprising said protein with first beads having coupled thereto an antibody directed against said protein,
   to form an antibody-protein complex,
(b) contacting said antibody-protein complex with one or more further beads, each further bead having coupled thereto (i) a label and (ii) a lectin,
   to form an antibody-protein-lectin complex; and
(c) determining the glycoprofile of said protein.
2. The method of item 1, further comprising step (d) comparing the glycoprofile of said protein with a control glycoprofile of said protein to determine whether the glycoprofile of said protein may deviate from the glycoprofile of said control glycoprofile.
3. The method of any one of the preceding items, further comprising step (a′) enriching said antibody-glycoprotein complex prior to step (b) contacting said antibody-glycoprotein complex with one or more further beads.
4. The method of any one of the preceding items, further comprising step (b′) enriching said antibody-protein-lectin complex prior to step (c) determining the glycoprofile of said protein.
5. The method of any one of the preceding items, wherein said protein is a cancer biomarker protein, an autoimmune disease biomarker protein, an inflammatory disease biomarker protein or a neurodegenerative disease biomarker protein.
6. The method of item 5, wherein said cancer biomarker protein is an ovarian cancer biomarker protein, breast cancer biomarker protein, colorectal cancer biomarker protein, pancreatic cancer biomarker protein, prostate cancer biomarker protein, thyroid cancer biomarker protein, liver cancer biomarker protein, lung cancer biomarker protein, stomach cancer biomarker protein, testicular cancer biomarker protein or bladder cancer biomarker protein.
7. The method of item 6, wherein prostate cancer biomarker protein is β-haptoglobin, TIMP-1, PSA, fPSA or tPSA.
8. The method of any one of the preceding items, wherein said lectin is specific for core fucose, antennary fucose, Fucα1-6GlcNAc-N-Asn containing N-linked oligosaccharides, Fucα1-6/3GlcNAc, α-L-Fuc, Fucα1-2Galβ1-4(Fucα1-3)GlcNAc, Fucα1-2Gal, Fucα1-6GlcNAc, Manβ1-4GlcNAcβ1-4GlcNAc, branched N-linked hexa-saccharide, Mana 1-3Man, α-D-Man, (GlcNAcβ1-4)₂₋₄, Galβ1-4GlcNAc, GlcNAcα1-4Galβ1-4GlcNAc, (GlcNAcβ1-4)₂₋₅, Neu5Ac (sialic acid), Galβ1-3GalNAc-serine/threonine, Galα1-3GalNAc, Galβ1-6Gal, Galβ1-4GlcNAc, Galβ1-3GalNAc, GalNAcα1-3GalNAc, GalNAcα1-3Gal, GalNAcα/β1-3/4Gal, α-GalNAc, GalNAcβ1-4Gal, GalNAcα1-3(Fucα1-2)Gal, GalNAcα1-2Gal, GalNAcα1-3GalNAc, GalNAcβ1-3/4Gal, GalNAc-Ser/Thr (Tn antigen), Galβ1-3GalNAc-Ser/Thr (T antigen), GalNAcβ1-4GlcNAc (LacdiNAc), α-2,3Neu5Ac (α2-3 linked sialic acid), α-2,6Neu5Ac (α2-6 linked sialic acid), α-2,8Neu5Ac (α2-8 linked sialic acid), sialic acid (α-2,3Neu5Ac, α-2,6Neu5Ac or α-2,8Neu5Ac), Neu5Acα4/9-O-Ac-Neu5Ac, Neu5Acα2-3Galβ1-4Glc/GlcNAc, Neu5Acα2-6Gal/GalNAc, N-linked bi-antennary, N-linked tri/tetra-antennary, branched β1-6GlcNAc, Galα1-3(Fucα1-2)Galβ1-3/4GlcNAc, Galβ1-3(Fucα1-4)GlcNAc, NeuAcα2-3Galβ1-3(Fucα1-4)GlcNAc, Fucα1-2Galβ1-3(Fucα1-4)GlcNAc, Galβ1-4(Fucα1-3)GlcNAc, NeuAcα2-3Galβ1-4(Fucα1-3)GlcNAc, Fucα1-2Galβ1-4(Fucα1-3)GlcNAc, high mannose, sialyl Lewis^{a} (sialyl Le^{a}) antigen, sialyl Lewis^{x} (sialyl Le^{x}) antigen, Lewis^{x} (Le^{x}) antigen, sialyl Tn antigen, sialyl T antigen, Lewis^{y} (Le^{y}) antigen, sulfated core1 glycan, Tn antigen, T antigen, core 2 glycan, Lewis^{a} (Le^{a}) antigen, (GlcNAcβ1-4)ₙ, β-D-GlcNAc, GalNAc, Gal-GlcNAc, GlcNAc, Galα1-3Gal, Galβ1-3GalNAc, α-Gal, α-GalNAc, (GlcNAc)ₙ, branched (LacNAc)ₙ.
9. A method for diagnosing whether a subject may be at a risk or may suffer from cancer, comprising
(a) contacting a sample obtained from said subject comprising a cancer biomarker protein with first beads having coupled thereto an antibody directed against said cancer biomarker protein, to form an antibody-protein complex,
(b) contacting said antibody-protein complex with one or more further beads, each further bead having coupled thereto (i) a label and (ii) a lectin, to form an antibody-protein-lectin complex; and
(c) determining the glycoprofile of said cancer biomarker protein,
   wherein a deviation of said glycoprofile from the healthy glycoprofile of said cancer biomarker protein is indicative that said subject may be at a risk or may suffer from cancer.
10. A method for diagnosing whether a subject may be at a risk or may suffer from an autoimmune disease, comprising
(a) contacting a sample obtained from said subject comprising an autoimmune disease biomarker protein with first beads having coupled thereto an antibody directed against said autoimmune disease biomarker protein, to form an antibody-protein complex,
(b) contacting said antibody-protein complex with one or more further beads, each further bead having coupled thereto (i) a label and (ii) a lectin, to form an antibody-protein-lectin complex; and
(c) determining the glycoprofile of said autoimmune disease biomarker protein,
   wherein a deviation of said glycoprofile from the healthy glycoprofile of said autoimmune disease biomarker protein is indicative that said subject may be at a risk or may suffer from an autoimmune disease.
11. A method for diagnosing whether a subject may be at a risk or may suffer from an inflammatory disease, comprising
(a) contacting a sample obtained from said subject comprising an inflammatory disease biomarker protein with first beads having coupled thereto an antibody directed against said inflammatory disease biomarker protein, to form an antibody-protein complex,
(b) contacting said antibody-protein complex with one or more further beads, each further bead having coupled thereto (i) a label and (ii) a lectin, to form an antibody-protein-lectin complex; and
(c) determining the glycoprofile of said inflammatory disease biomarker protein,
   wherein a deviation of said glycoprofile from the healthy glycoprofile of said inflammatory disease biomarker protein is indicative that said subject may be at a risk or may suffer from an inflammatory disease.
12. A method for diagnosing whether a subject may be at a risk or may suffer from a neurodegenerative disease, comprising
(a) contacting a sample obtained from said subject comprising a neurodegenerative disease biomarker protein with first beads having coupled thereto an antibody directed against said neurodegenerative disease biomarker protein, to form an antibody-protein complex,
(b) contacting said antibody-protein complex with one or more further beads, each further bead having coupled thereto (i) a label and (ii) a lectin, to form an antibody-protein-lectin complex; and
(c) determining the glycoprofile of said neurodegenerative disease biomarker protein,
   wherein a deviation of said glycoprofile from the healthy glycoprofile of said neurodegenerative disease biomarker protein is indicative that said subject may be at a risk or may suffer from a neurodegenerative disease.
13. A kit for performing the method of item 9, comprising an antibody specific for a cancer biomarker protein as defined in item 5 and one or more lectins as defined in item 8.
14. A kit for performing the method of item 10, comprising an antibody specific for an autoimmune disease biomarker protein which is IgG and one or more lectins as defined in item 8.
15. A kit for performing the method of item 11, comprising an antibody specific for an inflammatory biomarker protein which is IgG, IgA or CRP and one or more lectins as defined in item 8.
16. A kit for performing the method of item 12, comprising an antibody specific for a neurodegenerative biomarker protein, preferably α-synuclein, tau-protein or amyloid beta protein and its isoforms, and one or more lectins as defined in item 8.
17. The method of any one of the preceding items, wherein said first beads and said further beads are simultaneously brought into contact with said sample.
18. The method of any one of the preceding items, wherein said further beads are brought into contact with said sample immediately after said first beads were brought into contact with said sample.
19. The method of any one of the preceding items, wherein said first beads are brought into contact with said sample immediately after said second beads were brought into contact with said sample.
20. The method of any one of the preceding items, wherein said first bead and said further beads are in solution during performing the method of any one of the preceding items.
21. The method of any one of the preceding items, wherein said first bead and/or said further beads is/are made of glass, plastic, metal, agarose, latex, metallic nano- or microparticle, metal oxide nano- or microparticle or magnetic material.
22. The method of any one of the preceding items, wherein the label of said further beads is an enzyme, a radioisotope, a fluorescent protein, a fluorescent dye, a bioluminescent label or a tag (e.g., biotin).
23. The method of any one of the preceding items, wherein the label of said further beads is detected based on optical, fluorescent, luminescent, electrochemiluminescent and/or multi-analyte profiling (xMAP) readouts.
24. The method of any one of the preceding items, wherein for each of the one or more further beads for each carbohydrate detected by a lectin a different label is used in combination.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

As used herein the terms “about”, “approximately” or “essentially” mean within 20%, preferably within 15%, preferably within 10%, and more preferably within 5% of a given value or range. It also includes the concrete number, i.e. “about 20” includes the number of 20.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES

An even better understanding of the present invention and of its advantages will be evident from the following examples, offered for illustrative purposes only. The examples are not intended to limit the scope of the present invention in any way.

### Example 1

In this example, an application of two types of beads for cancer diagnostics based on glycoprofiling of proteins by analysis of glycans is described (see FIG. 1). Firstly, magnetic beads (“first beads”, or beads 1) are coupled to antibodies against a free form of PSA (fPSA) using standard bioconjugation protocols. The anti-fPSA antibody is selected in a way to bind to the epitope on fPSA, which is distant from the glycan epitope on fPSA (Asn61) occupied by N-glycan. The other type of beads (non-magnetic ones; i.e. “one or more further beads”, or beads 2) is coupled to a lectin able to selectively recognise cancer specific glycans using standard bioconjugation protocol. Lectins are co-immobilised together with the enzyme peroxidase, any other enzyme or any tag able to generate optical, fluorescent, chemiluminescent and electrochemiluminescent signal. Alternatively enzymes or tags are attached to beads 2 already modified by a lectin. In one approach the Inventors produce beads 2 modified by different enzyme and tags allowing us to analyse in a parallel/multiplex/array format of analysis.

Bead 1 coupled to anti-fPSA antibody is incubated with the sample containing fPSA allowed to react for substantial time, the complex is separated by a magnetic force and the complex attached to magnetic bead is washed by a washing buffer. In the next step the complex is incubated with beads 2 patterned by a lectin and a tag (enzyme or any other signal generating tag) and the overall complex is separated by magnetic force and washed by a washing buffer. The assay is performed in an ELISA plate, in any test tube in the solution phase or in the flow system of highly automatic machines. The assay can be repeated with additional one or several lectins. In such a format the assay shows increased sensitivity and specificity with a low background signal during analysis for diagnostics of prostate cancer. Further, the low limit of detection is confirmed. The method is applicable for glycoprofiling of any other glycoprotein produced by a prostate and any other organs and thus suitable for diagnostics of other cancer types.

### Example 2

The assay according to FIG. 2 uses two types of nanoparticles, namely integrated gold nanoparticles with a size of 40 nm and magnetic particles with the size of 130 nm. In this case, only the fPSA antibody was bound to the surface of the 130 nm magnetic particles (MNPs). Horseradish peroxidase (HRP) and lectin were bound to the surface of gold nanoparticles with a size of 40 nm, creating a sandwich configuration in presence of the analyte i.e. fPSA protein. The sandwich approach was subsequently used for the analysis of 110 real human sera samples. The results showed that fPSA glycoprofiling can provide much better clinical parameters compared to tPSA analysis (total PSA, prostate cancer screening biomarker). ROC (Receiver Operating Curve) curve showing the difference between tPSA (black line) and PGI+ index (light grey line) (FIG. 2) provided the following clinical parameters: AUC 0.886 (C195%=[0.581, 0.781], specificity=0.654, sensitivity=0.690, precision=0.673) for tPSA and 0.803 (C195%=[0.707, 0.890], specificity=0.712, sensitivity=0.828, precision=0.773) for PGH+ (gtycoprofiling (PSA).

1. A method of determining the glycoprofile of a protein, comprising
(a) contacting a sample comprising said protein with first beads having coupled thereto an antibody directed against said protein,
to form an antibody-protein complex,
(b) contacting said antibody-protein complex with one or more further beads, each further bead having coupled thereto (i) a label which amplifies a signal being generated and (ii) a lectin,
to form an antibody-protein-lectin complex; and
(c) determining the glycoprofile of said protein.

2. The method of claim 1, further comprising step (d) comparing the glycoprofile of said protein with a control glycoprofile of said protein to determine whether the glycoprofile of said protein may deviate from the glycoprofile of said control glycoprofile.

3. The method of any one of the preceding claims, further comprising
step (a′) enriching said antibody-glycoprotein complex prior to step (b) contacting said antibody-glycoprotein complex with one or more further beads; and/or
step (b′) enriching said antibody-protein-lectin complex prior to step (c) determining the glycoprofile of said protein.

4. The method of any one of the preceding claims, wherein said protein is a cancer biomarker protein, an autoimmune disease biomarker protein, an inflammatory disease biomarker protein or a neurodegenerative disease biomarker protein.

5. The method of any one of the preceding claims, wherein said lectin is specific for core fucose, antennary fucose, Fucα1-6GlcNAc-N-Asn containing N-linked oligosaccharides, Fucα1-6/3GlcNAc, α-L-Fuc, Fucα1-2Galβ1-4(Fucα1-3)GlcNAc, Fucα1-2Gal, Fucα1-6GlcNAc, Manβ1-4GlcNAcβ1-4GlcNAc, branched N-linked hexa-saccharide, Manα1-3Man, α-D-Man, (GlcNAcβ1-4)₂₋₄, Galβ1-4GlcNAc, GlcNAcα1-4Galβ1-4GlcNAc, (GlcNAcβ1-4)₂₋₅, Neu5Ac (sialic acid), Galβ1-3GalNAc-serine/threonine, Galα1-3GalNAc, Galβ1-6Gal, Galβ1-4GlcNAc, Galβ1-3GalNAc, GalNAcα1-3GalNAc, GalNAcα1-3Gal, GalNAcα/β1-3/4Gal, α-GalNAc, GalNAcβ1-4Gal, GalNAcα1-3(Fucα1-2)Gal, GalNAcα1-2Gal, GalNAcα1-3GalNAc, GalNAcβ1-3/4Gal, GalNAc-Ser/Thr (Tn antigen), Galβ1-3GalNAc-Ser/Thr (T antigen), GalNAcβ1-4GlcNAc (LacdiNAc), α-2,3Neu5Ac (α2-3 linked sialic acid), α-2,6Neu5Ac (α2-6 linked sialic acid), α-2,8Neu5Ac (α2-8 linked sialic acid), sialic acid (α-2,3Neu5Ac, α-2,6Neu5Ac or α-2,8Neu5Ac), Neu5Acα4/9-O-Ac-Neu5Ac, Neu5Acα2-3Galβ1-4Glc/GlcNAc, Neu5Acα2-6Gal/GalNAc, N-linked bi-antennary, N-linked tri/tetra-antennary, branched β1-6GlcNAc, Galα1-3(Fucα1-2)Galβ1-3/4GlcNAc, Galβ1-3(Fucα1-4)GlcNAc, NeuAcα2-3Galβ1-3(Fucα1-4)GlcNAc, Fucα1-2Galβ1-3(Fucα1-4)GlcNAc, Galβ1-4(Fucα1-3)GlcNAc, NeuAcα2-3Galβ1-4(Fucα1-3)GlcNAc, Fucα1-2Galβ1-4(Fucα1-3)GlcNAc, high mannose, sialyl Lewis^{a} (sialyl Le^{a}) antigen, sialyl Lewis^{x} (sialyl Le^{x}) antigen, Lewis^{x} (Le^{x}) antigen, sialyl Tn antigen, sialyl T antigen, Lewis^{y} (Le^{y}) antigen, sulfated core1 glycan, Tn antigen, T antigen, core 2 glycan, Lewis^{a} (Le^{a}) antigen, (GlcNAcβ1-4)ₙ, β-D-GlcNAc, GalNAc, Gal-GlcNAc, GlcNAc, Galα1-3Gal, Galβ1-3GalNAc, α-Gal, α-GalNAc, (GlcNAc)ₙ, branched (LacNAc)ₙ.

6. The method of claim 4, wherein the protein is a cancer biomarker protein and wherein a deviation of said glycoprofile from a healthy glycoprofile of said cancer biomarker protein is indicative that said subject may be at a risk or may suffer from cancer.

7. The method of claim 6, wherein said cancer biomarker protein is any one of an ovarian cancer biomarker protein, breast cancer biomarker protein, colorectal cancer biomarker protein, pancreatic cancer biomarker protein, prostate cancer biomarker protein, thyroid cancer biomarker protein, liver cancer biomarker protein, lung cancer biomarker protein, stomach cancer biomarker protein, testicular cancer biomarker protein or bladder cancer biomarker protein.

8. The method of claim 7, wherein the prostate cancer biomarker protein is any one of β-haptoglobin, TIMP-1, PSA, fPSA or tPSA.

9. The method of claim 4, wherein the protein is an autoimmune disease biomarker protein and wherein a deviation of said glycoprofile from a healthy glycoprofile of said autoimmune disease biomarker protein is indicative that said subject may be at a risk or may suffer from an autoimmune disease.

10. The method of claim 4, wherein the protein is an inflammatory disease biomarker protein and wherein a deviation of said glycoprofile from a healthy glycoprofile of said inflammatory disease biomarker protein is indicative that said subject may be at a risk or may suffer from an inflammatory disease.

11. The method of claim 4, wherein the protein is a neurodegenerative disease biomarker protein and wherein a deviation of said glycoprofile from a healthy glycoprofile of said neurodegenerative disease biomarker protein is indicative that said subject may be at a risk or may suffer from a neurodegenerative disease.

12. A kit for performing
(a) the method of claim 6, comprising an antibody specific for said cancer biomarker protein as defined in claim 7 and one or more lectins as defined in claim 5;
(b) the method of claim 9, comprising an antibody specific for said autoimmune disease biomarker protein which is IgG and one or more lectins as defined in claim 5;
(c) the method of claim 10, comprising an antibody specific for said inflammatory biomarker protein which is IgG, IgA or CRP and one or more lectins as defined in claim 5; or
(d) the method of claim 11, comprising an antibody specific for said neurodegenerative biomarker protein, which is α-synuclein, tau-protein or amyloid beta protein and its isoforms and one or more lectins as defined in claim 5.

13. The method of any one of claims 1-11,
(a) wherein said first beads and said further beads are simultaneously brought into contact with said sample;
(b) wherein said further beads are brought into contact with said sample immediately after said first beads were brought into contact with said sample; or
(c) wherein said first beads are brought into contact with said sample immediately after said second beads were brought into contact with said sample.

14. The method of any one of claims 1-11 and 13, wherein said first bead and said further beads are in solution during performing the method of any one of the preceding claims.

15. The method of any one of claims 1-11, 13 and 14, wherein said first bead and/or said further beads is/are made of glass, plastic, metal, agarose, latex, metallic nano- or microparticle, metal oxide nano- or microparticle or magnetic material.

16. The method of any one of claims 1-11 and 13-15,
(a) wherein the label of said further beads is an enzyme, a radioisotope, a fluorescent protein, a fluorescent dye, a bioluminescent label or a tag (e.g., biotin); and/or
(b) wherein the label of said further beads is detected based on optical, fluorescent, luminescent, electrochemiluminescent and/or multi-analyte profiling (xMAP) readouts.

17. The method of any one of claims 1-11 and 13-16, wherein for each of the one or more further beads for each carbohydrate detected by a lectin a different label is used in combination.


SEQUENCE LISTING (11 sequences)

SEQ ID NO: 1 - protein, 183 aa, Streptomyces avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 287 aa, Maackia amurensis
MATSNSKPTQVLLATFLTFFFLLLNNVNSSDELSFTINNFVPNEADLLFQGEASVSSTGVLQLTRVENGQPQKYSVGRALYAAPVRIWDNTTGSVASFSTSFTFVVKAPNPDITSDGLAFYLAPPDSQIPSGSVSKYLGLFNNSNSDSSNQIVAVELDTYFAHSYDPWDPNYRHIGIDVNGIESIKTVQWDWINGGVAFATITYLAPNKTLIASLVYPSNQTTFSVAASVDLKEILPEWVRVGFSAATGYPTEVETHDVLSWSFTSTLEANCDAATENNVHIARYTA

SEQ ID NO: 3 - protein, 290 aa, Canavalia ensiformis
MAISKKSSLFLPIFTFITMFLMVVNKVSSSTHETNALHFMFNQFSKDQKDLILQGDATTGTDGNLELTRVSSNGSPQGSSVGRALFYAPVHIWESSAVVASFEATFTFLIKSPDSHPADGIAFFISNIDSSIPSGSTGRLLGLFPDANVIRNSTTIDFNAAYNADTIVAVELDTYPNTDIGDPSYPHIGIDIKSVRSKKTAKWNMQNGKVGTAHIIYNSVDKRLSAVVSYPNADSATVSYDVDLDNVLPEWVRVGLSASTGLYKETNTILSWSFTSKLKSNEIPDIATVV

SEQ ID NO: 4 - protein, 237 aa, Canavalia ensiformis
ADTIVAVELDTYPNTDIGDPSYPHIGIDIKSVRSKKTAKWNMQNGKVGTAHIIYNSVDKRLSAVVSYPNADSATVSYDVDLDNVLPEWVRVGLSASTGLYKETNTILSWSFTSKLKSNSTHETNALHFMFNQFSKDQKDLILQGDATTGTDGNLELTRVSSNGSPQGSSVGRALFYAPVHIWESSAVVASFEATFTFLIKSPDSHPADGIAFFISNIDSSIPSGSTGRLLGLFPDAN

SEQ ID NO: 5 - protein, 313 aa, Aleuria aurantia
MPTEFLYTSKIAAISWAATGGRQQRVYFQDLNGKIREAQRGGDNPWTGGSSQNVIGEAKLFSPLAAVTWKSAQGIQIRVYCVNKDNILSEFVYDGSKWITGQLGSVGVKVGSNSKLAALQWGGSESAPPNIRVYYQKSNGSGSSIHEYVWSGKWTAGASFGSTVPGTGIGATAIGPGRLRIYYQATDNKIREHCWDSNSWYVGGFSASASAGVSIAAISWGSTPNIRVYWQKGREELYEAAYGGSWNTPGQIKDASRPTPSLPDTFIAANSSGNIDISVFFQASGVSLQQWQWISGKGWSIGAVVPTGTPAGW

SEQ ID NO: 6 - protein, 320 aa, Sambucus nigra
MRVIAAAMLYLYIVVLAICSVGIQGIDYPSVSFNLAGAKSATWDFLRMPHDLVGEDNKYNDGEPITGNIIGRDGLCVDVRNGYDTDGTPLQLWPCGTQRNQQWTFYTDDTIRSMGKCMTANGLSNGSNIMIFNCSTAVENAIKWEVTIDGSIINPSSGLVMTAPSAASFTILLLQNNIYAASQGWTVSNDVQPIVALIVGYNEMCLQSNGENNGVWMEDCEATSLQQQWALFGDRTIRVNSDRGLCVTTNGYNSKDLIIILQCQGLPSQRWFFNSNGAIVNPNSTLVMDVKESDVSLREIIIFPYHGDPNQQWVTQVLPS

SEQ ID NO: 7 - protein, 286 aa, Wisteria floribunda
MASSQTQNSFSVLLSISLTLFLLLLNKVNSKETTSFVFTRFSPDPQNLLLQGDTVVTSSGHLQLTQVKDGEPVYSSLGRALYYAPIHIWDSNTDTVANFVTSFSFVIDAPNKAKAADGLAFFLAPVDTEPQKPGGLLGLFHDDRHNKSNHIVAVEFDTFKNSWDPEGTHIGINVNSIVSRKTTSWDLENGEVANVVISYQASTKTLTASLVYPSSSTSYILNDVVDLKQILPEYVRVGFTAASGLSKDHVETHDVLAWTFDSDLPDPSSDDCNNLHLSSNVLRGSI

SEQ ID NO: 8 - protein, 11 aa, synthetic construct
  REGION 1..11: note = Microperoxidase (MP-11) peptide
VQKCAQCHTVE

SEQ ID NO: 9 - protein, 9 aa, synthetic construct
  REGION 1..9: note = microperoxidase-9 (MP-9) peptide
KCAQCHTVE

SEQ ID NO: 10 - protein, 8 aa, synthetic construct
  REGION 1..8: note = microperoxidase-8 (MP-8) peptide
CAQCHTVE

SEQ ID NO: 11 - protein, 105 aa, Equus caballus
MGDVEKGKKIFVQKCAQCHTVEKGGKHKTGPNLHGLFGRKTGQAPGFSYTDANKNKGITWKEETLMEYLENPKKYIPGTKMIFAGIKKKTEREDLIAYLKKATNE